SEQUENCE LISTING (61 sequences)

SEQ ID NO: 1 - protein, 114 aa, Trypanosoma brucei
PNLVVTATFSPPAFSIVGSPLREDTIKHMEERIPTVLSTALSRSREPSYFVHSKNPESWKMELSQHFCDELHRAAVSLVIIECLDAEGWVLRTSSSSQDTNEGKCCIKFYFTRS

SEQ ID NO: 2 - protein, 113 aa, Leishmania major
MPVIQTFVSTPLDHHKRENLAQVYRAVTRDVLGKPEDLVMMTFHDSTPMHFFGSTDPVACVRVEALGGYGPSEPEKVTSIVTAAITKECGIVADRIFVLYFSPLHCGWNGTNF

SEQ ID NO: 3 - protein, 116 aa, Trypanosoma cruzi
MPNLCVSATFNPPVITMLGSALREETVKLLEQRIPTGVSTSSSPSKDPVKFLFYPNPDHWRMELSQHFCDDLHKSAVFLTIIEGLEGEGWNLRASNSIRDSESGKDTTKLFFARRN

SEQ ID NO: 4 - protein, 116 aa, Trypanosoma cruzi
MPNLCVSATFNPPVITMLGSALREETVKLLEQRIPTDVSTSSSPSKDPVKFLFYTNPDHWRMELSQHFCDDLHKSAVFLTIIEGLEGEGWNLRASNSVRDSESGKDTTKLFFARRE

SEQ ID NO: 5 - protein, 116 aa, Trypanosoma rangeli
MPNLCVSATFNPPVITILGSALREETIKMMEQRIPASVSTSSSPSKEPIKFLFYPNPDHWRMELSQHFCNDLHKSAVFLTIIEALEGEGWNLRASSSTRDNDSGKDTTKLFFARGP

SEQ ID NO: 6 - protein, 115 aa, Leishmania braziliensis
MPNLCVSCTFNPPTITLLGSTIREDTVRAMETQIPLATSTAVNPSKDPAKFIFLSNPDHWRMELHQHFCDELHKSSVFLVIIQSLEQEGWRLRASNSVQNTENDTDTAKLFFVRA

SEQ ID NO: 7 - protein, 115 aa, Leishmania donovani
MPNLCVSCTFNPPTITLLGSTIREDTVRAMEAQLPLATSTAVNPSKDPAKFIFLSNPDHWRMDLNQHFCDELHKSSVFLVILQSLEEEGWRLRASNNVVNKENDRETVKLFFTRA

SEQ ID NO: 8 - protein, 115 aa, Leishmania infantum; identical to SEQ ID NO: 7

SEQ ID NO: 9 - protein, 115 aa, Leishmania mexicana
MPNLCVSCTFNPPSITLLGSTIREDTVRAMEAQLPLATSTAVNPSKDPVKFIFLSNPDHWRMDLNQHFCDELHKSSIFLVILQSLEEEGWRLRASNNVSRKENDGETVKLFFTRA

SEQ ID NO: 10 - protein, 115 aa, Leishmania major
MPNLCVSCTFNPPTITLLGSTIREDTVRAMEAQLPLATSTAVNPSKDPAKFIFLSNPDHWRMDLNQHFCDELHKSLVFLVILHSLEEEGWCLRASNNVSNKENDRETVKLFFTRS

SEQ ID NO: 11 - protein, 115 aa, Trypanosoma brucei
MPNLVVTATFSPPAFSIVGSPLREDTIKHMEERIPTVLSTALSRSREPSYFVHSKNPESWKMELSQHFCDELHRAAVSLVIIECLDAEGWVLRTSSSSQDTNEGKCCIKFYFTRS

SEQ ID NO: 12 - protein, 115 aa, Trypanosoma brucei; identical to SEQ ID NO: 11

SEQ ID NO: 13 - protein, 148 aa, Trypanosoma cruzi
MGAGRSVVRRLRNKCDYNKHRKLKKKKKKRRITMEKNLIINVTCNPPVISILGPVKESTIEKLNDVLPGACTTTNTGKVHFSFVRKEEPPHWYGELQTHFASEDIGQSQLFVSLLDAVEEEGMWKLCGSNANVHDYDKATHKFFFVRA

SEQ ID NO: 14 - protein, 140 aa, Angomonas deanei
MIQKPSTSLVSLLRLFLSNETSSMSELNLVVNITCNPPVISIYGPVRESTIDHLNDVLPGSCSTTNTGKVPFKLVRKDASCWHGELKSHFATNDIGTSHLFVSLLDALEEEGSWRLRGSTALNHDGNKCTYRFFFVRGAH

SEQ ID NO: 15 - protein, 120 aa, Trypanosoma cruzi
MPNVIVNCTFNPPLFSITGTQLRQETLDSLEKALPQCTTTSQNSMGAANGNAPKFMLVNSQPNTWRMPIGQHYCDQRGRCVVFSTIISTLQTEGWALRTTDSHESDNGKDITRLFFWRSG

SEQ ID NO: 16 - protein, 119 aa, Trypanosoma theileri
MPYVIVNCTFNPPTLSITGAQLRQETIDSLEKSIPQCTTTSSSSNSMNGTTAKFTLSESQPQTWHMSIGQQYCDQRGRCVVFAAVAQALQLEGWALRTTDAVSWENEKDVTRLFFWRSN

SEQ ID NO: 17 - protein, 125 aa, Trypanosoma brucei
MPVIVVNLVFNPPEVSLIGKGLSTSIMNRLESELIVRTTTSRPVTASMGTEGPLFVQNPNGCPAGAEVWRLTIPNHYCDDLAKCAFFGSVISVMEGDGWSLVCSSATTTSGDGKEISRFFFSRRA

SEQ ID NO: 18 - protein, 191 aa, Trypanosoma rangeli
MVLGYVRGHLVRVSPPLALLFSFSSSCVWELPLRVLHGAIANYKRKKNKQRRGSDGKSKTSTNNTDSSKPEMPSVIVNCTFNPPALSIVGAPLRQETLDALEKVLPCCTTTSQNSLNAAVGGAPKFMLVNNQPNTWRLSIGQHYCDQRGRCVMFAEIIKSLHAEGWTLRTTSSHESGNDKDVARLFFWRPS

SEQ ID NO: 19 - protein, 162 aa, Leishmania braziliensis
MPNVIVTCTFNPPSVTIQGASIRQDTIDALQRALPKQTTTSLPTQRPSEPAKFLLTSRSGTVQAGTVNRIEAGGAVADGVAEGREEFSSPNHNSTGVASGEDAEAYKSWHVDLGQHYCDQMGRTMIFLVIIEALEEEGFGLRGTHSLVVKEKDITRLIFVRS

SEQ ID NO: 20 - protein, 163 aa, Leishmania major
MPNVIVTCTFNPPCVSIQGANIRQDTIDALQRALPKQTTTSLPTQRPGEPAKFLLTNGSGVVESGTVNRIEDGSAAAGGAEEGSQENTSPHHSSHGVAPGEGAKEYKSWRIDLGQHYCDQTGRAMIFLVIIEALEEEGFALRGTHTLMMDREKDATQLIFVRS

SEQ ID NO: 21 - protein, 163 aa, Leishmania mexicana
MPNVIVTCTFNPPCVSIQGVNIRQDTIDALQRALPKQTTTSLPTQRPGEPAKFLLTSGGGVGQAGTVNRIEYGSAAAGGAEEGRQEATSPHNSSNGVAPGEGAKEYKSWRIDLGQHYCDQMGRAMIFLVIIEALEEEGFALRGTHTLVMEREKDTTQLIFVRS

SEQ ID NO: 22 - protein, 164 aa, Leptomonas pyrrhocoris
MPNVIVTCVFNPPTISIQGTSIRQDTIEALQRALPKQTTTSLPSQRPGESAKFLLTNGGGSRGAEGSAPHGATGGRIEDVADTEGRDNVRDGAAKTTDEVADNDKGYKSWRIDLGQHYCDQKGRSMIFLVIMEALEEEGFALRGTHTVVTEKEKDVTRLIFARA

SEQ ID NO: 23 - protein, 149 aa, Leptomonas seymouri
MPNVIVTCVFNPPTISIQGTSIRQDTIEALQRALPKQTTTSLPSQRPGESAKFLLTSGGATGGRIEGGADTGAHSNQDDSAAVKGEANNKGYNSWHIDLGQHYCDQKGRSMIFLVIMEALEEEGFALRGTHTVVTEKEKDVTRLIFSRA

SEQ ID NO: 24 - protein, 155 aa, Strigomonas culicis
MPHVIVTCLFNPPSISIQGASLRQDTIDALQRALPKHTTTSQLTYGRQEPPKFLLTASSAHPEAETGEKKEQEKETDATTAVSAALIVRDRVQNLEEYTSLKIELGKHYCCQKGRSMMFLAIMEALEEEGFAMRSTSAVTMDNGKDVTRLFFSRF

SEQ ID NO: 25 - protein, 126 aa, Trypanosoma cruzi
MVQNLVINLLFNSPEIRIMGPVKEQTIDKLNEVIPNATSTARSTRVAPSRFQYISNPNHWYMKLDGQFCDEDGISYLMVLLLDALEEEGLWKLVSSTALRTPVGQSSKDYTETHVLFMNKLVGDEI

SEQ ID NO: 26 - protein, 126 aa, Trypanosoma brucei
MSHYLLVTFTFNCPEIRILGPIKEQTIEKLNDVIPNATTTSRSNRTALPKFEYLPNPNHWYIKLDRQFCDEDGKSHLMVLLLDALSEEGSWRLVSSFVTKEPSGTGSKEGTETHTLFLNKLLAEDS

SEQ ID NO: 27 - protein, 126 aa, Leishmania infantum
MSRNMIVNMSFNCPEIQILGPVQESTIERLNEVLPDSTTSTRSIRSNQPKFEYLSNPDHWRIKLDNQFCDLEGVSRLIVLLLDALEEEGGWTLISSQASSSSHGGALQQDFVSNYKLFFSKYQDDE

SEQ ID NO: 28 - protein, 125 aa, Leishmania mexicana
MSHNMIVNMSFNCPEIQILGPVQESTIERLNEVLPDSTTSTRSIRSNQPKFEYVSNPDHWRIKLDNQFCDSEGVSRVIVCLLDALEEEGGWTLISSQASNSQGGSLQQDFMSNYKFFFSKYQDGE

SEQ ID NO: 29 - protein, 193 aa, Leishmania major
MAFVSVHEMALSHFSPAYVSRLFTDVIVSRPRSKLIAITLLPQAKQSPKIATPSKDYIKVHEPITCTMSRNMIVNMSFNSPEIQILGPVQESTIERLNEVLPDSTTSTRSIRSNQPKFEYVSNPDHWRIKLGNQFCDSEGVSRLIVLLLDALEEEGGWTLISSQASSSSHGGALQQDFVWNYKLFFSKYQDDE

SEQ ID NO: 30 - protein, 118 aa, Artificial Sequence
  Nb 27
QVQLQESGGGLVQPGGSLRLSCTTSRSILSIYAMGWYRQAPGKQRELVAVITSGGSTYYADSVKGRFTISRDNAKNTLYLQMNSLKPEDTALYYCARGGYGTSPQPSWGQGTQVTVSS

SEQ ID NO: 31 - protein, 119 aa, Artificial Sequence
  Nb 32
QVQLQESGGGLVQPGGSLRLSCAASGFTFSAYAMSWVRQAPGKGLEWVSTIDSGGGSTYYADSVKGRFTISRDNAKKTLYLHMNSLKPEDTAVYYCAKRGYSDYARHVWGQGTQVTVSS

SEQ ID NO: 32 - protein, 126 aa, Artificial Sequence
  Nb 34
QVQLQESGGGLVQPGGSLRLSCAASGFTFSNSPMNWVRQAPGKGLEWVSAINSGGGSTYYADSVKGRFTISRDNAKNTLYLQMNSLKPEDTAVYYCAKGGRTYYSGSYYSWGMDYWGKGTQVTVSS

SEQ ID NO: 33 - protein, 122 aa, Artificial Sequence
  Nb 36
QVQLQESGGGLVQPGGSLRLSCAASGFAFSFYWMNWVRQAPGKGLEWVSAINSGGGSTYYADSVKGRFTISRDNAKNTLYLQMNSLKPEDTAVYYCAKGIVQRGEYYGMDYWGKGTQVTVSS

SEQ ID NO: 34 - protein, 117 aa, Artificial Sequence
  Nb 39
QVQLQESGGGLVQPGGSLRLSCTASGSIFSGYAMGWYRQAPGKQRELVAGITSGGSTIYAGSVKGRFTISRDNAKNTVYLQMNSLKPEDTAVYYCKNGTYYSARDYWGQGTQVTVSS

SEQ ID NO: 35 - protein, 115 aa, Artificial Sequence
  Nb 41
QVQLQESGGGLVQAGGSLRLSCAASGRTFSSYAMGWFRQAPGKEREFIALISWIGGSTAYADSVKGRFTISRDNAKNTLYLQMNSLKPEDTAVYYCATDRIGRHRGPGTQVTVSS

SEQ ID NO: 36 - protein, 120 aa, Artificial Sequence
  Nb 53
QVQLQESGGGSVQAGGSLRLSCSASGYTYSSHCTAWFRQAPGKEREALAAINSRSGTTYYADSVKGRFTISQDNTKNTAYLQMNSLKPEDTATYFCAALSFGCTGPLASLGQGTQVTVSS

SEQ ID NO: 37 - protein, 120 aa, Artificial Sequence
  Nb 58
QVQLQESGGGLVQPGGSLRLSCAASGRSISSIYAMGWYRQAPGKQRELVARLTSGGSSNYADSVKGRFTITRDNAQNTVYLQMNSLKPEDTAVYYCNAEPSRWWLDDDYWGQGTQVTVSS

SEQ ID NO: 38 - protein, 8 aa, Artificial Sequence
  CDR
RSILSIYA

SEQ ID NO: 39 - protein, 7 aa, Artificial Sequence
  CDR
ITSGGST

SEQ ID NO: 40 - protein, 12 aa, Artificial Sequence
  CDR
ARGGYGTSPQPS

SEQ ID NO: 41 - protein, 8 aa, Artificial Sequence
  CDR
GFTFSAYA

SEQ ID NO: 42 - protein, 8 aa, Artificial Sequence
  CDR
IDSGGGST

SEQ ID NO: 43 - protein, 12 aa, Artificial Sequence
  CDR
AKRGYSDYARHV

SEQ ID NO: 44 - protein, 8 aa, Artificial Sequence
  CDR
GFTFSNSP

SEQ ID NO: 45 - protein, 8 aa, Artificial Sequence
  CDR
INSGGGST

SEQ ID NO: 46 - protein, 19 aa, Artificial Sequence
  CDR
AKGGRTYYSGSYYSWGMDY

SEQ ID NO: 47 - protein, 8 aa, Artificial Sequence
  CDR
GFAFSFYW

SEQ ID NO: 48 - protein, 8 aa, Artificial Sequence
  CDR
INSGGGST

SEQ ID NO: 49 - protein, 15 aa, Artificial Sequence
  CDR
AKGIVQRGEYYGMDY

SEQ ID NO: 50 - protein, 8 aa, Artificial Sequence
  CDR
GSIFSGYA

SEQ ID NO: 51 - protein, 7 aa, Artificial Sequence
  CDR
ITSGGST

SEQ ID NO: 52 - protein, 11 aa, Artificial Sequence
  CDR
KNGTYYSARDY

SEQ ID NO: 53 - protein, 8 aa, Artificial Sequence
  CDR
GRTFSSYA

SEQ ID NO: 54 - protein, 8 aa, Artificial Sequence
  CDR
ISWIGGST

SEQ ID NO: 55 - protein, 8 aa, Artificial Sequence
  CDR
ATDRIGRH

SEQ ID NO: 56 - protein, 8 aa, Artificial Sequence
  CDR
GYTYSSHC

SEQ ID NO: 57 - protein, 8 aa, Artificial Sequence
  CDR
INSRSGTT

SEQ ID NO: 58 - protein, 13 aa, Artificial Sequence
  CDR
AALSFGCTGPLAS

SEQ ID NO: 59 - protein, 9 aa, Artificial Sequence
  CDR
GRSISSIYA

SEQ ID NO: 60 - protein, 7 aa, Artificial Sequence
  CDR
LTSGGSS

SEQ ID NO: 61 - protein, 13 aa, Artificial Sequence
  CDR
NAEPSRWWLDDDY

(19) United States
(12) Patent Application Publication
(10) Pub. No.: US 2024/0156935 A1
(43) Pub. Date: May 16, 2024

(54) Vaccine Compositions for Trypanosomatids

(71) Applicants: VIB VZW, Gent (BE); Vrije Universiteit Brussel, Brussel (BE); Universiteit Antwerpen, Antwerpen (BE)

(72) Inventor: Benoit Stijlemans, Kampenhout (BE)

(21) Appl. No.: 18/552,827

(22) PCT Filed: Mar. 31, 2022
(86) PCT No.: PCT/EP2022/058575

§ 371 Date: Sep. 27, 2023

(30) Foreign Application Priority Data
Mar. 31, 2021 (EP) 21166420.6

(51) Int. Cl. A61K 39/005; A61K 39/00; A61K 39/002; A61K 39/008; A61P 37/04

(52) U.S. Cl. CPC A61K 39/005; A61K 39/002; A61K 39/008; A61P 37/04; A61K 2039/53

(57) ABSTRACT

The present invention provides vaccines and compositions, methods and uses of immunogenic vaccine compositions for eliciting an immune response to members of trypanosomatids such as Trypanosoma brucei, T. cruzi and Leishmania species.

### CROSS-REFERENCE TO RELATED APPLICATIONS

This application is a national phase entry under 35 U.S.C. § 371 of International Patent Application PCT/EP2022/058575, filed Mar. 31, 2022, designating the United States of America and published in English as International Patent Publication WO 2022/207793 on Oct. 6, 2022, which claims the benefit under Article 8 of the Patent Cooperation Treaty to European Patent Application Serial No. 21166420.6, filed Mar. 31, 2021, the entireties of which are hereby incorporated by reference.

### FIELD OF THE INVENTION

The present invention relates to the field of vaccines and particularly to compositions, methods and uses of immunogenic vaccine compositions for eliciting an immune response to members of trypanosomatids such as Trypanosoma brucei, T. cruzi and Leishmania species.

### INTRODUCTION TO THE INVENTION

African trypanosomes are extracellular flagellated protozoan parasites with a complex digenetic life cycle that can cause debilitating diseases of both medical and veterinary importance that can adversely influence the economic development of sub-Saharan Africa, see Buscher P et al (2017) Lancet 390:2397). Indeed, upon transmission through the bite of their blood-feeding vector (i.e., the tsetse fly, Glossina spp.), these parasites can cause fatal diseases in mammals, commonly called sleeping sickness in humans [Human African Trypanosomiasis (HAT)] or Nagana (AAT, Animal African Trypanosomiasis) in domestic livestock. As far as HAT is concerned, this is caused by either Trypanosoma brucei gambiense (accounting for over 95-97% of cases) and Trypanosoma brucei rhodesiense (accounting for the remainder of cases, see Simarro P P et al (2012) PLoS Negl Trop Dis 6: e1859). These diseases, exhibiting high morbidity and mortality rates, affect millions of impoverished populations in the developing world, display a limited response to chemotherapy, are unresponsive towards vaccination and are classified as neglected tropical diseases by the World Health Organization (WHO). On the other hand, Nagana (Trypanosoma brucei brucei, Trypanosoma congolense, Trypanosoma vivax) or Surra (Trypanosoma evansi) or Dourine (Trypanosoma equiperdum) are the typical forms of AAT, see Radwanska M. et al (2018) Front Immunol. 9:2253. Since, Nagana forms a major constraint on cattle production, it has a great impact on the nutrition of millions of people living in the most endemic areas, and on the agriculture economics of their countries, resulting in an estimated annual economic cost of about US$4 billion.

Due to millions of years of co-evolution, these parasites have been able to thwart host innate responses and escape early recognition, allowing the initiation of infection in their respective hosts. Indeed, these AT have developed efficient immune escape mechanisms to subvert the entire host immune response (cellular and humoral), involving an elaborate and efficient vector-parasite-host interplay, to survive sufficiently long in their mammalian host in order to complete their life cycle/transmission. Murine models which are more easily amenable compared to cattle or other domestic animals are very useful tools to study AT. Furthermore, given that the HAT causing T. b. rhodesiense and T. b. gambiense parasites highly resemble T. b. brucei (a non-human pathogenic subspecies causing Nagana), and chronic murine HAT models are scarce, the majority of research uses T. b. brucei as a model.

The efficient immune evasion mechanisms used by AT allows on the one hand persistence, yet on the other hand culminates into severe immunopathology (anemia and tissue injury). In this context, macrophage migration inhibitory factor (MIF) was shown to be a key host-derived molecule implicated in AT-associated immunopathology. Interestingly, most protozoan parasites including Plasmodium, Entamoeba, Toxoplasma, and Leishmania harbor a MIF-homologue which appears to function as a virulence factor aiding in the establishment or persistence of infection by modulating the host “innate” immune response. Of note, though the sequence identities between mammalian MIF and protozoan MIF ranges between 20-27%, they share a well conserved trimeric architecture/structure. Hence, AT might also harbor such a MIF-homologue thereby allowing modulating the host immune response. Surprisingly, though AT are closely related to Leishmania, and using the Leishmania MIF to blast the trypanosoma genome, we did not find a MIF-homologue in AT. However, in our search we identified a protein (Tb927.6.4140) which was annotated as a trypanosomatid-coding hypothetical protein with unknown function. Interestingly, when using the Phyre2 analysis software, we found homology with 4-oxalocrotonate isomerase (4-OT) at the first half of the Tb927.6.4140 protein sequence (with 51.5% confidence). 4-OT belongs to the tautomerase superfamily (TSF), to which also MIF belongs, whereby the TSF is characterized by the unusual and key catalytic role of an N-terminal proline and a structurally similar core domain that comprises a β-α-β motif. Based on the subgroups of the TSF it is speculated that MIF may have evolved by gene duplication and fusion from a simpler 4-OT-like ancestor. 4-OT is an ancestor protein playing a prominent role in the bacterial utilization of aromatic hydrocarbons as sole carbon sources. Hence, the protein (Tb927.6.4140) may be evolved from such an ancestor protein. To study its biological function, we purified the recombinant protein and generated nanobodies which allowed localization of the protein within the parasite as well as in different trypanosome ssp. and other trypanosomatids.

### SUMMARY

The present invention shows that Tb927.6.4140 and homologues thereof play a key role to allow early-stage infection and thus can be considered as a novel parasite-derived virulence factor. Moreover, functional in vitro assays revealed that recombinant Tb927.6.4140 activates myeloid cells to produce besides TNF, IL-6 and MIF also IL-10 further strengthening the notion that it is a regulatory molecule required to allow early parasite establishment. Finally, vaccination of mice with Tb927.6.4140 and subsequently challenged with several pathogens of the family of Trypanosomatidae were found to attenuate early parasite establishment and significantly prolong the survival time. We conclude that Tb927.6.4140 is considered a potential broad-spectrum vaccine for trypanosomatids.

### DESCRIPTION OF THE FIGURES

The patent or application file contains at least one drawing executed in color. Copies of this patent or patent application publication with color drawing(s) will be provided by the Office upon request and payment of the necessary fee.

The drawings described are only schematic and are non-limiting. In the drawings, the size of some of the elements may be exaggerated and not drawn on scale for illustrative purposes.

FIGS. 1A and 1B: Tb927.6.4140 is a highly conserved protein within trypanosomatids. The presence of Tb927.6.4140 homologues in trypanosomatids was evaluated using the https://www.ncbi.nlm.nih.gov/blast/treeview software. The protein sequences are identified, at left, by the species and their UniProt code. Secondary structure elements of Q4D6Q6 as reference, as determined experimentally, are depicted schematically on top. Sequences ID NO: 3-29 are in the sequence listing.

FIGS. 2A-2C: Evolutionary relationships of Tb927.6.4140. The depicted tree represents a consensus phylogram obtained by Bayesian analysis of the amino-acid sequences of confirmed Tb927.6.4140 proteins of a wide range of eukaryotes. Branch lengths are proportional to the estimated number of substitutions per site (see scale bar). Numbers above branches represent Bayesian posterior probabilities; only branches with values >0.95 should be regarded as highly supported. Branches with probabilities <0.5 are collapsed. A sequence alignment was created using Mafft 7 and entered into MrBayes 3.2.6 for Bayesian phylogenetic inference. A mixed prior implementing multiple empirical models of amino-acid substitution was applied, in combinations with gamma-correction for among-site rate heterogeneity and an estimated proportion of invariable sites. Two parallel runs of four incrementally heated (temperature parameter=0.2) Markov chain Monte Carlo (MCMC) chains were performed, with a length of 6,000,000 generations, a sampling frequency of 1 per 1000 generations, and a burn-in corresponding to the first 1,000,000 generations. Convergence of the parallel runs was confirmed by split frequency standard deviations (<0.01) and potential scale reduction factors (approximating 1.0) for all model parameters, as reported by MrBayes. Adequate posterior sampling was verified using Tracer 1.6, by checking if the runs had reached effective sampling sizes >200 for all model parameters.

FIGS. 3A-3C: Purity of anti-Q586B2 Nbs and binding to recombinant Tb927.6.4140 FIG. 3A) Sequence alignment of the different anti-Q586B2 Nbs, which are organized into different families according to Kabat classification (Sequences ID NO: 30 to 37 are in the sequence listing). Complementarity determinant region 1 (CDR1), CDR2, and CDR3 are highlighted in grey). FIG. 3B) 12% SDS page showing the purity of the different anti-Q586B2 Nbs following purification via IMAC and SEC (3 ul Nb/lane). FIG. 3C) Titration curves of the different anti-Q586B2 Nbs on coated Q586B2 Affinity measurement and estimated KD values of the different Nbs.

FIGS. 4A and 4B: Binding profiles of anti-Q586B2 40 Nbs on purified T. brucei brucei parasites via FACS and localization of Q586B2 via immunofluorescence microscopy. FIG. 4A) Representative flow cytometric profile of purified, fixed and permeabilized T. brucei brucei parasites plotted in an SSC-A versus FSC-A plot and subsequently gating on single cells (FSC-H versus FSC-A plot). 10⁶ parasites in presence or absence of 5 μg of HA-tagged Nb was used per condition. Detection of the Nbs was achieved using an Alexa-488 labelled anti-HA IgG (1 μg). Signals of parasites in presence of these Alexa-488 labelled anti-HA IgG alone was used as negative control, while Nb33 (anti-VSG Nb) was used as a positive control. FIG. 4B) Immunofluorescence microscopy showing representative images of purified, fixed and permeabilized T. brucei brucei parasites stained with dapi (i.e. nucleus) alone or in presence of anti-Q586B2 Nbs (Nb27 and Nb39) or an anti-VSG Nb (Nb33).

FIGS. 5A-5C: Binding profiles of the anti-Q586B2 Nb39 on purified trypanosomatid parasites via FACS. Representative flow cytometric anti-Q586B2 Nb binding profile on different purified, fixed and permeabilized parasites (Gating as described in FIG. 3A). Per condition, 10⁶ purified parasites were used in presence or absence of 5 μg of HA-tagged Nb39. Detection of the Nbs was achieved using an Alexa-488 labelled anti-HA IgG (1 μg). FIG. 5A) Parasites used T. b. brucei bloodstream form (AnTat1.1E), procyclic form and metacyclic form. Signals of parasites without staining (red), parasites in presence of the Alexa-488 labelled anti-HA IgG alone (blue) and parasites in presence of Nb39 and Alexa-488 labelled anti-HA IgG (orange) are shown. FIG. 5B) Parasites used T. b. brucei, T. b. gambiense, T. b. rhodesiense, T. evansi, T. congolense, and T. vivax. FIG. 5C) Parasites used Leishmania Major, Leishmania Infantum and T. cruzi. Signals of parasites in presence of the Alexa-488 labelled anti-HA IgG alone was used as negative control (black/grey).

FIG. 6: Q586B2 is released by parasites and exerts both pro- and anti-inflammatory activity on myeloid cells (BMDMs) in vitro. A) Production of TNF, IL-6, MIF and IL-10 by BMDMs generated from C57BL/6 mice (2*10⁵ cells/well) following 48 hours incubation with a ½ serial dilution of LPS-free Q586B2 (white bars) starting from 0.5 μg/ml or buffer alone (black bars). Negative controls: cells incubated with buffer alone or without any protein. Data are shown as means±SEM and representative of 2 independent experiments (n=3). *P<0.05, **P<0.01, ***P<0.001, ****P<0.0001.

FIG. 7: Q586B2 exerts both pro- and anti-inflammatory activity on myeloid cells (BMDMs) in vitro. Production of cytokines by BMDMs generated from C57BL/6 mice (2*10⁵ cells/well) following 48 hours incubation with 0.5 μg/ml of LPS-free Q586B2 (white bars) or buffer alone (black bars). Negative controls: cells incubated with buffer alone or without any protein. Data are shown as means±SEM and representative of 2 independent experiments (n=3). *P<0.05, ****P<0.0001.

FIG. 8: Q586B2 triggers the induction of IL-10 mainly by myeloid cells at the site of inoculation (within the peritoneum) and myeloid cells mediate the attenuates first peak parasitemia observed in Tb927.6.4140-KO parasite infected mice. To assess which cells express IL-10 in vivo, IL-10-GFP (VeRT-X) mice were injected intraperitoneally with PBS (control) or 5 μg LPS-free Q586B2 and 18 hours later sacrificed. Subsequently, the cellular source of IL-10 was analyzed via flow cytometry using the gating strategy described in FIG. 7. The expression of IL-10 was presented as delta median fluorescence intensity (delta MFI), by subtracting the MFI of PBS injected mice from Q586B2 injected mice, for each of the different immune cells that were identified. Results are representative of 2 independent experiments and presented as mean of 2-3 individual mice±SEM. ND: Not detected.

FIGS. 9A-9E: Anti-Q586B2 Nbs exhibit a diagnostic potential and exert a Q586B2 blocking potential on myeloid cells (BMDMs) in vitro, while Q586B2 can be considered a broad spectrum prophylactic therapeutic for African trypanosomes. FIG. 9A) Detection of native Q586B2 via a Nb-based sandwich ELISA in serum from naïve and T. b. brucei infected C57BL/6 mice collected at day 6 p.i. Nb39 was used as capturing Nb and biotinylated Nb-39 was used as detecting Nbs. Streptavidin-HRP was used to develop the ELISA. FIG. 9B) BMDMs generated from C57BL/6 mice (2*10⁵ cells/well) were incubated with 1 μg/ml of LPS-free Q586B2 (white bars) or buffer alone (black bars in presence or absence of anti-Q586B2 Nbs (5 μg/ml). After 48 hours incubation the culture medium was tested for IL-10 production. Negative controls: cells incubated with buffer alone or without any protein. Dashed red line represent levels of cytokines produced by BMDMs stimulated with Q586B2 alone and all groups were compared to this signal. Data are shown as means±SEM and representative of 2 independent experiments (n=3). *P<0.05. FIG. 9C) Parasitemia development and survival of mock-treated (black circles) and Q586B2-treated (white circles) C57BL/6 mice infected with Wild type T. b. brucei parasites. FIG. 9D) Parasitemia development and survival of mock-treated (black circles) and Q586B2-treated (white circles) C57BL/6 mice infected with T. congolense parasites. FIG. 9E) Parasitemia development and survival of mock-treated (black circles) and Q586B2-treated (white circles) C57BL/6 mice infected with T. evansi parasites. Results are presented as mean (parasitemia) or median (survival) of 5 individual mice±SEM. **: p≤0.01, ***: p≤0.001.

FIGS. 10A-10D: Q586B2 treatment triggers a strong anti-Q586B2 humoral response challenging of Q586B2 treated mice attenuates first peak parasitemia and prolongs survival in B-cell deficient (μMT) mice. FIG. 10A) Antibody (IgG) response of Q586B2-treated mice. FIG. 10B) Anti-Q586B2 IgG recognizes the native protein in lysate of T. b. brucei parasites in ELISA. FIG. 10C) Parasitemia and FIG. 10D) survival of mock-treated (black circles) and Q586B2-treated (white circles) μMT mice infected with T. b. brucei parasites. Results are presented as mean (parasitemia) or median (survival) of 5 individual mice±SEM. *: p≤05, **: p≤0.01, ***: p≤001.

### DETAILED DESCRIPTION OF THE INVENTION

Throughout the specification and claims, the following terms take the meanings explicitly associated herein, unless the context clearly dictates otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

To facilitate an understanding of the present technology, a number of terms and phrases are defined below. Additional definitions are set forth throughout the detailed description.

### Definitions

As used herein, the terms “subject” and “patient” refer to any animal, such as a mammal like a dog, cat, bird, livestock, and preferably a human.

As used herein, the term “pharmaceutical composition” refers to the combination of an active agent with a carrier, inert or active, making the composition especially suitable for therapeutic use.

The terms “pharmaceutically acceptable” or “pharmacologically acceptable”, as used herein, refer to compositions that do not substantially produce adverse reactions, e.g., toxic, allergic, or immunological reactions, when administered to a subject.

As used herein, the term “treating” includes reducing or alleviating at least one adverse effect or symptom of a disease or disorder through introducing in any way a therapeutic composition of the present technology into or onto the body of a subject. “Treatment” refers to both therapeutic treatment and prophylactic or preventative measures, wherein the object is to prevent or slow down (e.g., minimize or lessen) the targeted pathologic condition or disorder. Those in need of treatment include those already with the disorder as well as those prone to have the disorder or those in whom the disorder is to be prevented.

As used herein, the term “antibody” is used in its broadest sense to refer to whole antibodies, monoclonal antibodies (including human, humanized, or chimeric antibodies), polyclonal antibodies, and antibody fragments that can bind antigen (e.g., Fab′, F′(ab)2, FV, single chain antibodies), comprising complementarity determining regions (CDRs) of the foregoing as long as they exhibit the desired biological activity. Particularly preferred herein are immunoglobulin single domain antibodies such as VHH's or nanobodies.

A molecule that “specifically binds to” or is “specific for” another molecule is one that binds to that particular molecule without substantially binding to any other molecule.

As used herein the term, “in vitro” refers to an artificial environment and to processes or reactions that occur within an artificial environment. In vitro environments may include, but are not limited to, test tubes and cell cultures. The term “in vivo” refers to the natural environment (e.g., an animal or a cell) and to processes or reactions that occur within a natural environment.

As used herein, the term “administration” refers to the act of giving a drug, prodrug, antibody, vaccine, or other agent, or therapeutic treatment to a physiological system (e.g., a subject or in vivo, in vitro, or ex vivo cells, tissues, and organs). Exemplary routes of administration to the human body can be through the eyes (ophthalmic), mouth (oral), skin (transdermal), nose (nasal), lungs (inhalant), oral mucosa (buccal), ear, by injection (e.g., intravenously, subcutaneously, intratumorally, intraperitoneally, etc.) and the like.

“Co-administration” refers to administration of more than one chemical agent or therapeutic treatment (e.g. anti-trypanosomatid drugs such as for example nifurtimox, melarsoprol, suramin, eflornithine, pentamidine, pentostam and fexinidazole) to a physiological system (e.g., a subject or in vivo, in vitro, or ex vivo cells, tissues, and organs). As used herein, administration “in combination with” one or more further therapeutic agents includes simultaneous (concurrent) and consecutive administration in any order. “Coadministration” of therapeutic treatments may be concurrent, or in any temporal order or physical combination.

As used herein, “carriers” include pharmaceutically acceptable carriers, excipients, or stabilizers which are nontoxic to the cell or mammal being exposed thereto at the dosages and concentrations employed. Often the physiologically acceptable carrier is an aqueous pH-buffered solution. Examples of physiologically acceptable carriers include buffers such as phosphate, citrate, and other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine, or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants.

As used herein, the terms “protein,” “polypeptide,” and “peptide” refer to a molecule comprising amino acids joined via peptide bonds. In general, “peptide” is used to refer to a sequence of 20 or less amino acids and “polypeptide” is used to refer to a sequence of greater than 20 amino acids.

As used herein, the term, “synthetic polypeptide,” “synthetic peptide”, and “synthetic protein” refer to peptides, polypeptides, and proteins that are produced by a recombinant process (i.e., expression of exogenous nucleic acid encoding the peptide, polypeptide, or protein in an organism, host cell, or cell-free system) or by chemical synthesis.

As used herein, the term “protein of interest” refers to a protein encoded by a nucleic acid of interest. In the present invention the protein of interest is a protein depicted by SEQ ID NO: 1 or a protein with an amino acid length of at least 48% over the total length of SEQ ID NO: 1.

As used herein, the term “native” (or wild type) when used in reference to a protein refers to proteins encoded by the genome of a cell, tissue, or organism, other than one manipulated to produce synthetic proteins.

As used herein, “domain” (typically a sequence of three or more, generally 5 or 7 or more amino acids) refers to a portion of a molecule, such as proteins or the encoding nucleic acids, that is structurally and/or functionally distinct from other portions of the molecule and is identifiable. For example, domains include those portions of a polypeptide chain that can form an independently folded structure within a protein made up of one or more structural motifs and/or that is recognized by virtue of a functional activity, such as proteolytic activity. As such, a domain refers to a folded protein structure that retains its tertiary structure independently of the rest of the protein. Generally, domains are responsible for discrete functional properties of proteins, and in many cases may be added, removed or transferred to other proteins without loss of function of the remainder of the protein and/or of the domain.

As used herein, the term “host cell” refers to any eukaryotic cell (e.g., mammalian cells, avian cells, amphibian cells, plant cells, fish cells, insect cells, yeast cells), and bacteria cells, and the like, whether located in vitro or in vivo (e.g., in a transgenic organism). The term “host cell” refers to any cell capable of replicating and/or transcribing and/or translating a heterologous gene. Thus, a “host cell” refers to any eukaryotic or prokaryotic cell, whether located in vitro or in vivo. For example, host cells may be located in a transgenic animal.

As used herein, the term “cell culture” refers to any in vitro culture of cells. Included within this term are continuous cell lines (e.g., with an immortal phenotype), primary cell cultures, finite cell lines (e.g. non-transformed cells), and any other cell population maintained in vitro, including oocytes and embryos.

The term “isolated” when used in relation to a nucleic acid or polypeptide or protein refers to a nucleic acid or polypeptide or protein sequence that is identified and separated from at least one contaminant nucleic acid or polypeptide or protein with which it is ordinarily associated in its natural source. Isolated nucleic acids or polypeptides or proteins are molecules present in a form or setting that is different from that in which they are found in nature. In contrast, non-isolated nucleic acids or polypeptides or proteins are found in the state in which they exist in nature.

The term “antigen” refers to a molecule (e.g., a protein, glycoprotein, lipoprotein, lipid, nucleic acid, or other substance) that is reactive with an antibody specific for a portion of the molecule. In the present invention the antigen is depicted in SEQ ID NO: 1 or a protein with an amino acid identity of at least 48% with SEQ ID NO: 1 (or an amino acid identity of at least 48% over the total length of SEQ ID NO: 1).

The term “antigenic determinant” refers to that portion of an antigen that makes contact with a particular antibody (e.g., an epitope). When a protein or fragment of a protein is used to immunize a host animal, numerous regions of the protein may induce the production of antibodies that bind specifically to a given region or three-dimensional structure on the protein; these regions or structures are referred to as antigenic determinants. An antigenic determinant may compete with the intact antigen (e.g., the “immunogen” used to elicit the immune response) for binding to an antibody.

The terms “protein” and “polypeptide” refer to compounds comprising amino acids joined via peptide bonds and are used interchangeably. A “protein” or “polypeptide” encoded by a gene is not limited to the amino acid sequence encoded by the gene but includes post-translational modifications of the protein.

Where the term “amino acid sequence” is recited herein to refer to an amino acid sequence of a protein molecule, “amino acid sequence” and like terms, such as “polypeptide” or “protein” are not meant to limit the amino acid sequence to the complete, native amino acid sequence associated with the recited protein molecule. Furthermore, an “amino acid sequence” can be deduced from the nucleic acid sequence encoding the protein.

The term “portion” when used in reference to a protein (as in “a portion of a given protein”) refers to fragments of that protein. The fragments may range in size from four amino acid residues to the entire amino sequence minus one amino acid (for example, the range in size includes 4, 5, 6, 7, 8, 9, 10, or 11 . . . amino acids up to the entire amino acid sequence minus one amino acid).

As used herein, a “vaccine” comprises one or more immunogenic antigens intentionally administered to induce acquired immunity in the recipient (e.g., a subject).

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms also encompass “consisting of” and “consisting essentially of”, which enjoy well-established meanings in patent terminology.

The recitation of numerical ranges by endpoints includes all numbers and fractions subsumed within the respective ranges, as well as the recited endpoints. This applies to numerical ranges irrespective of whether they are introduced by the expression “from . . . to . . . ” or the expression “between . . . and . . . ” or another expression.

The terms “about” or “approximately” as used herein when referring to a measurable value such as a parameter, an amount, a temporal duration, and the like, are meant to encompass variations of and from the specified value, such as variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” or “approximately” refers is itself also specifically, and preferably, disclosed.

The discussion of the background to the invention herein is included to explain the context of the invention. This is not to be taken as an admission that any of the material referred to was published, known, or part of the common general knowledge in any country as of the priority date of any of the claims. Throughout this disclosure, various publications, patents and published patent specifications are referenced by an identifying citation. All documents cited in the present specification are hereby incorporated by reference in their entirety. In particular, the teachings or sections of such documents herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the invention. When specific terms are defined in connection with a particular aspect of the invention or a particular embodiment of the invention, such connotation or meaning is meant to apply throughout this specification, i.e., also in the context of other aspects or embodiments of the invention, unless otherwise defined.

In the following passages, different aspects or embodiments of the invention are defined in more detail. Each aspect or embodiment so defined may be combined with any other aspect(s) or embodiment(s) unless clearly indicated to the contrary. In particular, any feature indicated as being preferred or advantageous may be combined with any other feature or features indicated as being preferred or advantageous.

Reference throughout this specification to “one embodiment”, “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the appended claims, any of the claimed embodiments can be used in any combination.

Practitioners are particularly directed to Sambrook et al., Molecular Cloning: A Laboratory Manual, 4th ed., Cold Spring Harbor Press, Plainsview, New York (2012); and Ausubel et al., current Protocols in Molecular Biology (Supplement 100), John Wiley & Sons, New York (2012), for definitions and terms of the art. The definitions provided herein should not be construed to have a scope less than understood by a person of ordinary skill in the art.

In the present invention we have identified and characterized a protein (i.e. Tb927.6.4140 (also designated herein as Q586B2), of which the sequence is depicted in SEQ ID NO: 1) that shows similarity with 4-oxalocrotonate isomerase (4-01) which is pant of the tautomerase superfamily, and which is not present in mammals. Using a nanobody-based approach we found that the protein was localized intracellular in vesicles, expressed during all life stages of T. brucei, and present in all trypanosome ssp. tested, indicating that it is a conserved protein within trypanosomes. Moreover, it was also present in T. cruzi, L. major and L. infantum, yet, surprisingly not in Plasmodium falciparum, indicating that is a relatively conserved protein within the trypanosomatids. In the present invention we have shown that Tb927.6.4140 plays a key role in the early stage of infection, which was reflected by the fact that immunized mice with this protein show a greatly reduced first peak parasitemia and a significantly prolonged survival upon challenge with T. brucei, T. evansi an T. congolense. In addition, this protein was shown to induce besides TNF-alpha, MIF and IL-6 by mouse myeloid cells also IL-10 in vitro, suggesting that it could be a regulatory molecule important for early parasite establishment. Thus, the invention shows that Tb927.6.4140 is an important secreted parasite-derived virulence factor essential for early-stage infection within the mammalian host. Given that it is highly conserved within trypanosomatids suggests that it is an essential molecule and that there is an evolutionary selection for this family of proteins that may represent a potential virulence factor in trypanosomatids. In addition, Tb927.6.4140 can be considered a potential diagnostic and therapeutic target for trypanosomatids in general.

The sequence of Tb927.6.4140 (alternative name is Q586B2, origin: Trypanosoma brucei brucei) is depicted in SEQ ID NO: 1 below:

NH₂-
PNLVVTATFSPPAFSIVGSPLREDTIKHMEERIPTVLSTALSRSREPSY
FVHSKNPESWKMELSQHFCDELHRAAVSLVIIECLDAEGWVLRTSSSSQ
DTNEGKCCIKFYFTRS-COOH

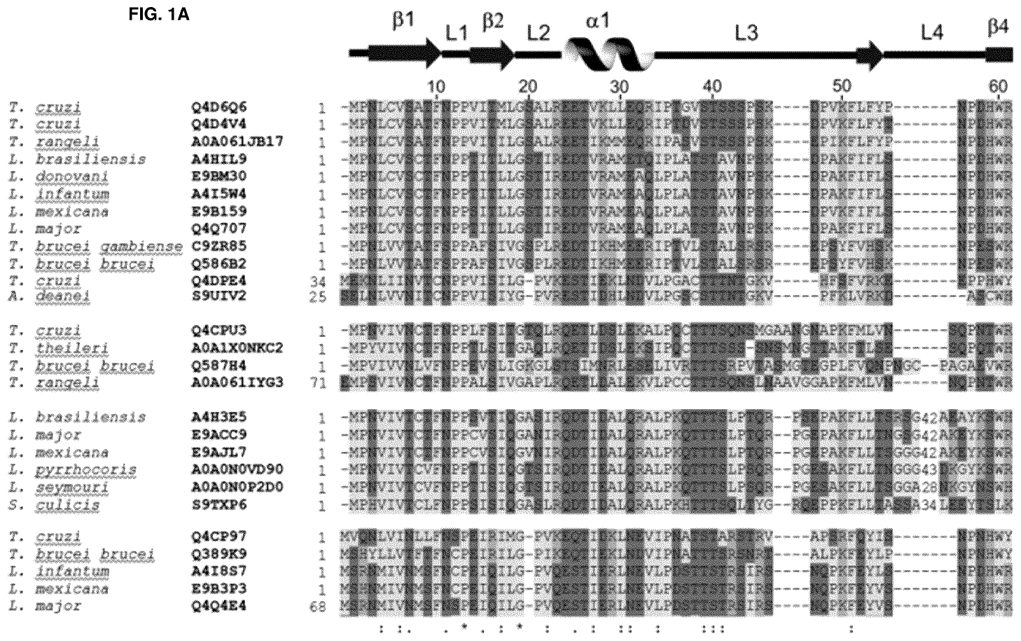

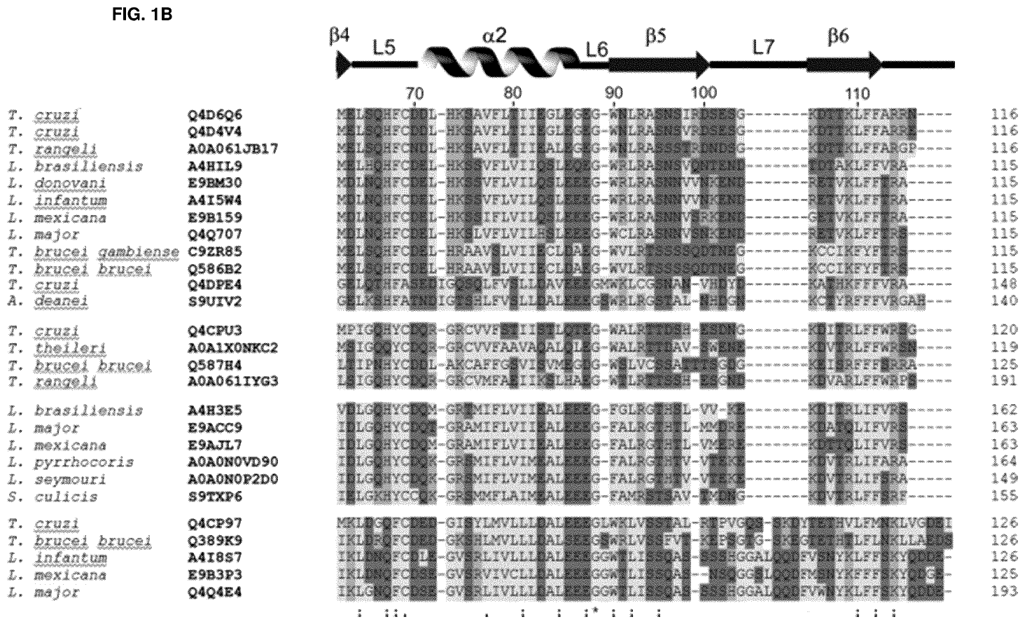

FIG. 1 provides a list of orthologous sequences derived from trypanosomatids (SEQ ID NO: 3 to 29). The orthologous protein sequences depicted in FIG. 1 have an amino acid sequence identity of at least 48% over the total length of SEQ ID NO: 1. These orthologous amino acid sequences belong to species from the family of Trypanosomatidae. In the present invention the species belonging to the family of Trypanosomatidae is also designated as trypanosomatids.

Therefore, the invention provides in a first embodiment a vaccine composition comprising a protein depicted in SEQ ID NO: 1 and an adjuvant or said composition comprises a protein with an amino acid identity of at least 48% over the length of SEQ ID NO: 1 and an adjuvant.

In yet another embodiment the invention provides a vaccine composition comprising a protein depicted in SEQ ID NO: 1 or a protein with an amino acid identity of at least 48%, at least 50% identity, at least 55% identity, at least 60% identity, at least 65% identity, at least 70% identity, at least 80% identity, at least 90% identity, at least 95% identity with SEQ ID NO: 1 and an adjuvant.

In a particular embodiment the vaccine composition comprises any of the proteins or combination of proteins selected from the list depicted in SEQ ID NO: 3 to 29 and an adjuvant.

In a preferred embodiment the vaccine composition comprises any of the proteins or combination of proteins selected from the list protein depicted in SEQ ID NO: 4, 8, 10, 11, 12, 13, 15, 17, 20, 25, 26, 27 or 29 and an adjuvant.

In another embodiment the invention provides a vaccine composition for use to treat or to prevent (or protect) mammals against infection of species of the family Trypanosomatidae, said composition comprising a protein depicted in SEQ ID NO: 1 and an adjuvant or said composition comprises a protein with an amino acid identity of at least 48% over the total length of SEQ ID NO: 1 and an adjuvant.

In a particular embodiment the vaccine composition comprises a nucleic acid sequence capable of encoding a protein having an amino acid identity of at least 48% over the length of SEQ ID NO: 1.

In another particular embodiment the nucleic acid sequence is DNA or RNA.

In another particular embodiment the vaccine composition comprises a pharmaceutically acceptable carrier.

In another particular embodiment the invention provides vaccine composition as described herein before for use to treat or to prevent an infection in a mammal from a species of the family Trypanosomatidae which species is selected from the genera Trypanosoma and Leishmania.

In a particular embodiment the Trypanosoma species is Trypanosoma brucei gambiense, Trypanosoma brucei brucei, Trypanosoma brucei rhodesiense, Trypanosoma congolense, Trypanosoma vivax; Trypanosoma evansi, Trypanosoma equiperdum or Trypanosoma cruzi.

In another particular embodiment the Leishmania species is Leishmania major or Leishmania infantum.

### Adjuvants

Adjuvants are described in general in Vaccine Design the Subunit and Adjuvant Approach, edited by Powell and Newman, Plenum Press, New York, 1995, incorporated by reference herein in its entirety for all purposes. Yet another useful resource of vaccine adjuvants is Vaxjo which is a vaccine adjuvant database (see the weblinks disclosed in Sayers S et al (2012) J. of Biomedicine and Biotechnology Article ID 831486). Importantly the present invention is not limited by the type of adjuvant utilized (e.g., for use in a composition (e.g. a pharmaceutical composition)). For example, in some embodiments, suitable adjuvants include an aluminium salt such as aluminium hydroxide gel (e.g., alum) or aluminium phosphate. In some embodiments, an adjuvant may be a salt of calcium, iron, or zinc, or it may be an insoluble suspension of acylated tyrosine, or acylated sugars, cationically or anionically derivatized polysaccharides, or polyphosphazenes.

In general, an immune response is generated to an antigen through the interaction of the antigen with the cells of the immune system. Immune responses may be broadly categorized into two categories: humoral and cell-mediated immune responses (e.g., traditionally characterized by antibody and cellular effector mechanisms of protection, respectively). These categories of response have been termed Th1-type responses (cell-mediated response), and Th2-type immune responses (humoral response).

Stimulation of an immune response can result from a direct or indirect response of a cell or component of the immune system to an intervention (e.g., exposure to an antigenic unit). Immune responses can be measured in many ways including activation, proliferation, or differentiation of cells of the immune system (e.g., B cells, T cells, dendritic cells, APCs, macrophages, NK cells, NKT cells etc.); up-regulated or down-regulated expression of markers and cytokines; stimulation of IgA, IgM, or IgG titre; splenomegaly (including increased spleen cellularity); hyperplasia and mixed cellular infiltrates in various organs. Other responses, cells, and components of the immune system that can be assessed with respect to immune stimulation are known in the art.

Although an understanding of the mechanism is not necessary to practice the present invention and the present invention is not limited to any particular mechanism of action, in some embodiments, compositions and methods of the present invention induce expression and secretion of cytokines (e.g., by macrophages, dendritic cells and CD4+ T cells). Modulation of expression of a particular cytokine can occur locally or systemically. It is known that cytokine profiles can determine T cell regulatory and effector functions in immune responses. In some embodiments, Th1-type cytokines can be induced, and thus, the immunostimulatory compositions of the present invention can promote a Th1-type antigen-specific immune response including cytotoxic T-cells (e.g., thereby avoiding unwanted Th2 type immune responses (e.g., generation of Th2 type cytokines (e.g., IL-13) involved in enhancing the severity of disease (e.g., IL-13 induction of mucus formation). Cytokines play a role in directing the T cell response. Helper (CD4+) T cells orchestrate the immune response of mammals through production of soluble factors that act on other immune system cells, including B and other T cells. Most mature CD4+T helper cells express one of two cytokine profiles: Th1 or Th2. Th1-type CD4+ T cells secrete IL-2, IL-3, IFN-γ, GM-CSF and high levels of INF-α. Th2 cells express IL-3, IL-4, IL-5, IL-6, IL-9, IL-10, IL-13, GM-CSF, and low levels of TNF-α. Th1 type cytokines promote both cell-mediated immunity and humoral immunity that is characterized by immunoglobulin class switching to IgG2a in mice and IgG1 in humans. Th1 responses may also be associated with delayed-type hypersensitivity and autoimmune disease. Th2 type cytokines induce primarily humoral immunity and induce class switching to IgG1 and IgE. The antibody isotypes associated with Th1 responses generally have neutralizing and opsonizing capabilities whereas those associated with Th2 responses are associated more with allergic responses.

Several factors have been shown to influence skewing of an immune response towards either a Th1 or Th2 type response. The best characterized regulators are cytokines. IL-12 and IFN-γ are positive Th1 and negative Th2 regulators. IL-12 promotes IFN-γ production, and IFN-γ provides positive feedback for IL-12. IL-4 and IL-appear important for the establishment of the Th2 cytokine profile and to down-regulate Th1 cytokine production.

Thus, in preferred embodiments, the present invention provides a method of stimulating a Th1-type immune response in a subject comprising administering to a subject a composition comprising an antigenic unit (e.g., a protein depicted in SEQ ID NO:1 or a protein with an amino acid identity of at least 48% identity with SEQ ID NO: 1 as described herein). However, in other embodiments, the present invention provides a method of stimulating a Th2-type immune response in a subject (e.g., if balancing of a T cell mediated response is desired) comprising administering to a subject a composition comprising an antigenic unit (e.g. a protein depicted in SEQ ID NO:1 or a protein with an amino acid identity of at least 48% identity with SEQ ID NO: 1). In further preferred embodiments, adjuvants can be used (e.g., can be co-administered with a composition of the present invention) to skew an immune response toward either a Th1 or Th2 type immune response. For example, adjuvants that induce Th2 or weak Th1 responses include, but are not limited to, alum, saponins, and SB-As4. Adjuvants that induce Th1 responses include but are not limited to MPL, MDP, ISCOMS, IL-12, IFN-γ and SB-A52.

Several other types of Th1-type immunogens can be used (e.g. as an adjuvant) in compositions and methods of the present invention. These include, but are not limited to, the following. In some embodiments, monophosphoryl lipid A (e.g., in particular, 3-de-O-acylated monophosphoryl lipid A (3D-MPL)), is used.

In some embodiments, saponins are used as an immunogen (e.g. Th1-type adjuvant) in a composition of the present invention. Saponins are well known adjuvants (See, e.g., Lacaille-Dubois and Wagner (1996) Phytomedicine vol 2 pp 363-386). Examples of saponins include Quil A (derived from the bark of the South American tree Quillaja Saponaria Molina), and fractions thereof (See, e.g., U.S. Pat. No. 5,057,540; Kensil, Crit Rev Ther Drug Carrier Syst, 1996, 12 (1-2): 1-55; and EP 03622.79, each of which is hereby incorporated by reference in its entirety). Also contemplated to be useful in the present invention are the haemolytic saponins QS7, QS17, and QS21 (HPLC purified fractions of Quil A; See, e.g., Kensil et al. (1991). J. Immunology 146, 431-437, U.S. Pat. No. 5,057,540; WO 96/33739; WO 96/11711 and EP 0 362 279, each of which is hereby incorporated by reference in its entirety). Also contemplated to be useful are combinations of QS21 and polysorbate or cyclodextrin (See, e.g., WO 99/10008, hereby incorporated by reference in its entirety).

In some embodiments, an immunogenic oligonucleotide containing unmethylated CpG dinucleotides (“CpG”) is used as an adjuvant. CpG is an abbreviation for cytosine-guanosine dinucleotide motifs present in DNA. CpG is known in the art as being an adjuvant when administered by both systemic and mucosal routes (See, e.g., WO 96/02555, EP 468520, Davis et al, J. Immunol, 1998, 160(2): 870-876; McCluskie and Davis, J. Immunol, 1998, 161(9):4463-6; and U.S. Pat. App. No. 20050238660, each of which is hereby incorporated by reference in its entirety). For example, in some embodiments, the immunostimulatory sequence is Purine-Purine-C-G-pyrimidine-pyrimidine; wherein the CG motif is not methylated.

Although an understanding of the mechanism is not necessary to practice the present invention and the present invention is not limited to any particular mechanism of action, in some embodiments, the presence of one or more CpG oligonucleotides activates various immune subsets including natural killer cells (which produce IFN-γ) and macrophages. In some embodiments, CpG oligonucleotides are formulated into a composition of the present invention for inducing an immune response. In some embodiments, a free solution of CpG is co-administered together with an antigen (e.g., present within a solution (See, e.g., WO 96/02555; hereby incorporated by reference). In some embodiments, a CpG oligonucleotide is covalently conjugated to an antigen (See, e.g., WO 98/16247, hereby incorporated by reference), or formulated with a carrier such as aluminium hydroxide (See, e.g., Brazolot-Millan et al, Proc. Natl, Acad. Sci., USA, 1998, 95(26), 15553-8).

In some embodiments, adjuvants such as Complete Freunds Adjuvant and Incomplete Freunds Adjuvant, cytokines (e.g., interleukins (e.g., IL-2, IFN-γ, IL-4, etc.), macrophage colony stimulating factor, tumor necrosis factor, etc.), detoxified mutants of a bacterial ADP-ribosylating toxin such as a cholera toxin (CT), a pertussis toxin (PT), or an E. coli heat-labile toxin (LT), particularly LT-K63 (where lysine is substituted for the wild-type amino acid at position 63), LT-R72 (where arginine is substituted for the wild-type amino acid at position 72), CT-S109 (where serine is substituted for the wild-type amino acid at position 109), and PT-K9/G129 (where lysine is substituted for the wild-type amino acid at position 9 and glycine substituted at position 129) (see, e.g., WO93/13202 and WO92/19265, each of which is hereby incorporated by reference), and other immunogenic substances (e.g., that enhance the effectiveness of a composition of the present invention) are used with a composition comprising a protein depicted in SEQ ID NO:1 or a protein with an amino acid identity of at least 48% identity with SEQ ID NO: 1.

Additional examples of adjuvants that find use in the present invention include poly(di(carboxylatophenoxy)phosphazene (PCPP polymer; Virus Research Institute, USA); derivatives of lipopoly saccharides such as monophosphoryl lipid A (MRL; Ribi ImmunoChem Research, Inc., Hamilton, Mont.), murarnyl dipeptide (MDP; Ribi) and threonyl-murarnyl dipeptide (t-MDP; Ribi); OM-174 (a glucosamine disaccharide related to lipid A; OM Pharma SA, Meyrin, Switzerland); and Leishmania elongation factor (a purified Leishmania protein; Corixa Corporation, Seattle, Wash.).

Adjuvants may be added to a composition comprising a protein depicted in SEQ ID NO:1 or a protein with an amino acid identity of at least 48% identity with SEQ ID NO: 1, or the adjuvant may be formulated with carriers, for example liposomes or metallic salts (e.g., aluminium salts (e.g., aluminium hydroxide)) prior to combining with or co-administration with a composition.

In some embodiments, a composition comprising a protein depicted in SEQ ID NO:1 or a protein with an amino acid identity of at least 48% identity with SEQ ID NO: 1 comprises a single adjuvant. In other embodiments, a composition comprises two or more adjuvants.

In some embodiments, a composition comprising a protein depicted in SEQ ID NO:1 or a protein with an amino acid identity of at least 48% identity with SEQ ID NO: 1 comprises one or more mucoadhesives (See, e.g., U.S. Pat. App. No. 20050281843, hereby incorporated by reference in its entirety). The present invention is not limited by the type of mucoadhesive utilized. Indeed, a variety of mucoadhesives is contemplated to be useful in the present invention including, but not limited to, cross-linked derivatives of poly(acrylic acid) (e.g., carbopol and polycarbophil), polyvinyl alcohol, polyvinyl pyrollidone, polysaccharides (e.g., alginate and chitosan), hydroxypropyl methylcellulose, lectins, fimbria) proteins, and carboxymethylcellulose. Although an understanding of the mechanism is not necessary to practice the present invention and the present invention is not limited to any particular mechanism of action, in some embodiments, use of a mucoadhesive (e.g., in a composition comprising a protein depicted in SEQ ID NO:1 or a protein with an amino acid identity of at least 48% identity with SEQ ID NO: 1) enhances induction of an immune response in a subject (e.g., administered a composition of the present invention) due to an increase in duration and/or amount of exposure to an antigenic unit that a subject experiences when a mucoadhesive is used compared to the duration and/or amount of exposure to a protein depicted in SEQ ID NO:1 or a protein with an amino acid identity of at least 48% identity with SEQ ID NO: 1 in the absence of using the mucoadhesive.

In some embodiments, a composition of the present invention may comprise sterile aqueous preparations. Acceptable vehicles and solvents include, but are not limited to, water, Ringer's solution, phosphate buffered saline, and isotonic sodium chloride solution. In addition, sterile, fixed oils are conventionally employed as a solvent or suspending medium. In addition, fatty acids such as oleic acid find use in the preparation of injectables, Carrier formulations suitable for mucosal, subcutaneous, intramuscular, intraperitoneal, intravenous, or administration via other routes may be found in Remington's Pharmaceutical Sciences, Mack Publishing Company, Easton, Pa.

A composition comprising a protein depicted in SEQ ID NO:1 or a protein with an amino acid identity of at least 48% identity with SEQ ID NO: 1 of the present invention can be used therapeutically (e.g., to enhance an immune response) or as a prophylactic (e.g., for immunization (e.g., to prevent signs or symptoms of disease)). A composition comprising a protein depicted in SEQ ID NO:1 or a protein with an amino acid identity of at least 48% identity with SEQ ID NO: 1 of the present invention can be administered to a subject via a number of different delivery routes and methods.

For example, the compositions of the present invention can be administered to a subject (e.g., mucosally (e.g., nasal mucosa, vaginal mucosa, etc.)) by multiple methods, including, but not limited to: being suspended in a solution and applied to a surface; being suspended in a solution and sprayed onto a surface using a spray applicator; being mixed with a mucoadhesive and applied (e.g., sprayed or wiped) onto a surface (e.g., mucosal surface); being placed on or impregnated onto a nasal and/or vaginal applicator and applied; being applied by a controlled-release mechanism; being applied as a liposome; or being applied on a polymer.

In some embodiments, compositions of the present invention are administered mucosally (e.g., using standard techniques; See, e.g., Remington: The Science and Practice of Pharmacy, Mack Publishing Company, Easton, Pa., 19th edition, 1995 (e.g., for mucosal delivery techniques, including intranasal, pulmonary, vaginal, and rectal techniques), as well as European Publication No. 517,565 and Ilium et al, J. Controlled Rel., 1994, 29:133-141 (e.g., for techniques of intranasal administration), each of which is hereby incorporated by reference in its entirety). Alternatively, the compositions of the present invention may be administered dermally or trans dermally using standard techniques (See, e.g., Remington: The Science arid Practice of Pharmacy, Mack Publishing Company, Easton, Pa., 19th edition, 1995). The present invention is not limited by the route of administration.

Although an understanding of the mechanism is not necessary to practice the present invention and the present invention is not limited to any particular mechanism of action, in some embodiments, mucosal vaccination is the route of administration as it has been shown that mucosal administration of antigens induces protective immune responses at mucosal surfaces (e.g., mucosal immunity), the route of entry of many pathogens. In addition, mucosal vaccination, such as intranasal vaccination, may induce mucosal immunity not only in the nasal mucosa, but also in distant mucosal sites such as the genital mucosa (See, e.g., Mestecky, Journal of Clinical Immunology, 7:265-276, 1987). In addition to inducing mucosal immune responses, mucosal vaccination also induces systemic immunity. In some embodiments, non-parenteral administration (e.g., mucosal administration of vaccines) provides an efficient and convenient way to boost systemic immunity (e.g., induced by parenteral or mucosal vaccination (e.g., in cases where multiple boosts are used to sustain a vigorous systemic immunity)).

In some embodiments, a composition comprising a protein depicted in SEQ ID NO:1 or a protein with an amino acid identity of at least 48% identity with SEQ ID NO: 1 of the present invention may be used to protect or treat a subject susceptible to, or suffering from, a trypansomatid disease by means of administering a composition of the present invention via a mucosal route (e.g., an oral/alimentary or nasal route). Alternative mucosal routes include intravaginal and intra-rectal routes. In some embodiments of the present invention, a nasal route of administration is used, termed “intranasal administration” or “intranasal vaccination” herein. Methods of intranasal vaccination are well known in the art, including the administration of a droplet or spray form of the vaccine into the nasopharynx of a subject to be immunized. In some embodiments, a nebulized or aerosolized composition is provided. Enteric formulations such as gastro resistant capsules for oral administration, suppositories for rectal or vaginal administration also form part of this invention. Compositions of the present invention may also be administered via the oral route. Under these circumstances, a composition comprising a protein depicted in SEQ ID NO:1 or a protein with an amino acid identity of at least 48% identity with SEQ ID NO: 1 may comprise a pharmaceutically acceptable excipient and/or include alkaline buffers or enteric capsules, Formulations for nasal delivery may include those with dextran or cyclodextran and saponin as an adjuvant.

Compositions of the present invention may also be administered via a vaginal route. In such cases, a composition comprising a protein depicted in SEQ ID NO:1 or a protein with an amino acid identity of at least 48% identity with SEQ ID NO: 1 may comprise pharmaceutically acceptable excipients and/or emulsifiers, polymers (e.g., CARBOPOL), and other known stabilizers of vaginal creams and suppositories. In some embodiments, compositions of the present invention are administered via a rectal route. In such cases, compositions may comprise excipients and/or waxes and polymers known in the art for forming rectal suppositories.

In some embodiments, the same route of administration (e.g., mucosal administration) is chosen for both a priming and boosting vaccination. In some embodiments, multiple routes of administration are utilized (e.g., at the same time, or, alternatively, sequentially) in order to stimulate an immune response.

For example, in some embodiments, a composition comprising a protein depicted in SEQ ID NO:1 or a protein with an amino acid identity of at least 48% identity with SEQ ID NO: 1 is administered to a mucosal surface of a subject in either a priming or boosting vaccination regime. Alternatively, in some embodiments, the composition is administered systemically in either a priming or boosting vaccination regime. In some embodiments, a composition comprising a protein depicted in SEQ ID NO:1 or a protein with an amino acid identity of at least 48% identity with SEQ ID NO: 1 is administered to a subject in a priming vaccination regimen via mucosal administration and a boosting regimen via systemic administration. In some embodiments, a composition comprising a protein depicted in SEQ ID NO:1 or a protein with an amino acid identity of at least 48% identity with SEQ ID NO: 1 is administered to a subject in a priming vaccination regimen via systemic administration and a boosting regimen via mucosal administration. Examples of systemic routes of administration include, but are not limited to, a parenteral, intramuscular, intradermal, transdermal, subcutaneous, intraperitoneal, or intravenous administration. A composition comprising a protein depicted in SEQ ID NO:1 or a protein with an amino acid identity of at least 48% identity with SEQ ID NO: 1 may be used for both prophylactic and therapeutic purposes.

In some embodiments, compositions of the present invention are administered by pulmonary delivery. For example, a composition of the present invention can be delivered to the lungs of a subject (e.g., a human) via inhalation (e.g., thereby traversing across the lung epithelial lining to the blood stream. Further contemplated for use in the practice of this invention is a wide range of mechanical devices designed for pulmonary and/or nasal mucosal delivery of pharmaceutical agents including, but not limited to, nebulizers, metered dose inhalers, and powder inhalers, all of which are familiar to those skilled in the art. Typically, each formulation is specific to the type of device employed and may involve the use of an appropriate propellant material, in addition to the usual diluents, adjuvants, surfactants, carriers, and/or other agents useful in therapy. Also, the use of liposomes, microcapsules or microspheres, inclusion complexes, or other types of carriers is contemplated.

Thus, in some embodiments, a composition comprising a protein depicted in SEQ ID NO:1 or a protein with an amino acid identity of at least 48% identity with SEQ ID NO: 1 of the present invention may be used to protect and/or treat a subject susceptible to, or suffering from, a disease caused by a trypanosomatid by means of administering the composition by mucosal, intramuscular, intraperitoneal, intradermal, transdermal, pulmonary, intravenous, subcutaneous or other route of administration described herein. Methods of systemic administration of the vaccine preparations may include conventional syringes and needles, or devices designed for ballistic delivery of solid vaccines, or needleless pressure liquid jet device or transdermal patches. The present invention may also be used to enhance the immunogenicity of antigens applied to the skin (transdermal or transcutaneous delivery. Thus, in some embodiments, the present invention provides a delivery device for systemic administration, pre-filled with the vaccine composition of the present invention.

The present invention is not limited by the type of subject administered (e.g., in order to stimulate an immune response (e.g., in order to generate protective immunity (e.g., mucosal and/or systemic immunity)) a composition of the present invention. Indeed, a wide variety of subjects are contemplated to be benefited from administration of a composition of the present invention. In preferred embodiments, the subject is a human. In some embodiments, human subjects are of any age (e.g., adults, children, infants, etc.) that have been or are likely to become exposed to a trypanosomatid species (e.g., T. brucei gambiense). In some embodiments, the human subjects are subjects that are more likely to receive a direct exposure to pathogenic microorganisms or that are more likely to display signs and symptoms of disease after exposure to a trypanosomatid pathogen (e.g., immune suppressed subjects). In some embodiments, the general public is administered (e.g., vaccinated with) a composition of the present invention (e.g., to prevent the occurrence or spread of disease). For example, in some embodiments, compositions and methods of the present invention are utilized to vaccinate a group of people (e.g., a population of a region, city, state and/or country) for their own health (e.g., to prevent or treat disease). In some embodiments, the subjects are non-human mammals (e.g., pigs, cattle, goats, horses, sheep, or other livestock, or other animal). In some embodiments, compositions and methods of the present invention are utilized in research settings (e.g., with research animals).

A composition of the present invention may be formulated for administration by any route, such as mucosal, oral, transdermal, intranasal, parenteral or other route described herein. The compositions may be in any one or more different forms including, but not limited to, tablets, capsules, powders, granules, lozenges, foams, creams or liquid preparations.

Topical formulations of the present invention may be presented as, for instance, ointments, creams or lotions, foams, and aerosols, and may contain appropriate conventional additives such as preservatives, solvents (e.g., to assist penetration), and emollients in ointments and creams.

Topical formulations may also include agents that enhance penetration of the active ingredients through the skin. Exemplary agents include a binary combination of N-(hydroxy ethyl) pyrrolidone and a cell-envelope disordering compound, a sugar ester in combination with a sulfoxide or phosphine oxide, and sucrose monooleate, decyl methyl sulfoxide, and alcohol.

In certain embodiments of the invention, compositions may further comprise one or more alcohols, zinc-containing compounds, emollients, humectants, thickening and/or gelling agents, neutralizing agents, and surfactants. Water used in the formulations is preferably deionized water having a neutral pH. Additional additives in the topical formulations include, but are not limited to, silicone fluids, dyes, fragrances, pH adjusters, and vitamins.

Topical formulations may also contain compatible conventional carriers, such as cream or ointment bases and ethanol or oleyl alcohol for lotions. Such carriers may be present as from about 1% up to about 98% of the formulation. The ointment base can comprise one or more of petrolatum, mineral oil, ceresin, lanolin alcohol, panthenol, glycerin, bisabolol, cocoa butter and the like.

In some embodiments, pharmaceutical compositions of the present invention may be formulated and used as foams. Pharmaceutical foams include formulations such as, but not limited to, emulsions, microemulsions, creams, jellies, and liposomes. While basically similar in nature these formulations vary in the components and the consistency of the final product.

The compositions of the present invention may additionally contain other adjunct components conventionally found in pharmaceutical compositions. Thus, for example, the compositions may contain additional, compatible, pharmaceutically-active materials such as, for example, antipruritics, astringents, local anesthetics, or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, preferably do not unduly interfere with the biological activities of the components of the compositions of the present invention. The formulations can be sterilized and, if desired, mixed with auxiliary agents (e.g., lubricants, preservatives, stabilizers, wetting agents, emulsifiers, salts for influencing osmotic pressure, buffers, colorings, flavorings and/or aromatic substances and the like) that do not deleteriously interact with the antigenic unit or other components of the formulation. In some embodiments, immunostimulatory compositions of the present invention are administered in the form of a pharmaceutically acceptable salt. When used, the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

In some embodiments, vaccine compositions are co-administered with one or more anti-trypanosomatid medicines.

The present invention also includes methods involving co-administration of a vaccine composition comprising a protein depicted in SEQ ID NO:1 or a protein with an amino acid identity of at least 48% identity with SEQ ID NO: 1 with one or more additional active and/or immunostimulatory agents (e.g., a composition comprising a different antigenic unit, an antibiotic, anti-oxidant, etc.). Indeed, it is a further aspect of this invention to provide methods for enhancing prior art immunostimulatory methods (e.g., immunization methods) and/or pharmaceutical compositions by co-administering a composition of the present invention. In coadministration procedures, the agents may be administered concurrently or sequentially. In one embodiment, the compositions described herein are administered prior to the other active agent(s). The pharmaceutical formulations and modes of administration may be any of those described herein. In addition, the two or more co-administered agents may each be administered using different modes (e.g., routes) or different formulations. The additional agents to be co-administered (e.g., antibiotics, anti-trypanosomatid medicines, adjuvants, etc.) can be any of the well-known agents in the art, including, but not limited to, those that are currently in clinical use.

In some embodiments, a composition comprising a protein depicted in SEQ ID NO:1 or a protein with an amino acid identity of at least 48% identity with SEQ ID NO:1 is administered to a subject via more than one route. For example, a subject that would benefit from having a protective immune response (e.g., immunity) towards a pathogenic microorganism may benefit from receiving mucosal administration (e.g., nasal administration or other mucosal routes described herein) and, additionally, receiving one or more other routes of administration (e.g., parenteral or pulmonary administration (e.g., via a nebulizer, inhaler, or other methods described herein). In some preferred embodiments, administration via mucosal route is sufficient to induce both mucosal as well as systemic immunity towards an antigenic unit or organism from which the antigenic unit is derived. In other embodiments, administration via multiple routes serves to provide both mucosal and systemic immunity. Thus, although an understanding of the mechanism is not necessary to practice the present invention and the present invention is not limited to any particular mechanism of action, in some embodiments, it is contemplated that a subject administered a composition of the present invention via multiple routes of administration (e.g., immunization (e.g., mucosal as well as airway or parenteral administration of the composition) may have a stronger immune response to an antigenic unit than a subject administered a composition via just one route.

Other delivery systems can include time-release, delayed release, or sustained release delivery systems. Such systems can avoid repeated administrations of the compositions, increasing convenience to the subject and a physician. Many types of release delivery systems are available and known to those of ordinary skill in the art.

In some embodiments, a vaccine composition of the present invention is formulated in a concentrated dose that can be diluted prior to administration to a subject. For example, dilutions of a concentrated composition may be administered to a subject such that the subject receives any one or more of the specific dosages provided herein. In some embodiments, dilution of a concentrated composition may be made such that a subject is administered (e.g., in a single dose) a composition comprising 0.5-50% of an emulsion and antigenic unit present in the concentrated composition. Concentrated compositions are contemplated to be useful in a setting in which large numbers of subjects may be administered a composition of the present invention (e.g., an immunization clinic, hospital, school, etc.). In some embodiments, a composition comprising a protein depicted in SEQ ID NO:1 or a protein with an amino acid identity of at least 48% identity with SEQ ID NO: 1 of the present invention (e.g., a concentrated composition) is stable at room temperature for more than 1 week, in some embodiments for more than 2 weeks, in some embodiments for more than 3 weeks, in some embodiments for more than 4 weeks, in some embodiments for more than 5 weeks, and in some embodiments for more than 6 weeks.

In some embodiments, following an initial administration of a composition of the present invention (e.g., an initial vaccination), a subject may receive one or more boost administrations (e.g., around 2 weeks, around 3 weeks, around 4 weeks, around 5 weeks, around 6 weeks, around 7 weeks, around 8 weeks, around 10 weeks, around 3 months, around 4 months, around 6 months, around 9 months, around 1 year, around 2 years, around 3 years, around 5 years, around 10 years) subsequent to a first, second, third, fourth, fifth, sixth, seventh, eighth, ninth, tenth, and/or more than tenth administration. Although an understanding of the mechanism is not necessary to practice the present invention and the present invention is not limited to any particular mechanism of action, in some embodiments, reintroduction of an antigenic unit in a boost dose enables vigorous systemic immunity in a subject. The boost can be with the same formulation given for the primary immune response or can be with a different formulation that contains the antigenic unit. The dosage regimen will also, at least in part, be determined by the need of the subject and be dependent on the judgment of a practitioner.

Dosage units may be proportionately increased or decreased based on several factors including, but not limited to, the weight, age, and health status of the subject. In addition, dosage units may be increased or decreased for subsequent administrations (e.g., boost administrations).

It is contemplated that the compositions and methods of the present invention will find use in various settings, including research settings. For example, compositions and methods of the present invention also find use in studies of the immune system (e.g., characterization of adaptive immune responses (e.g., protective immune responses (e.g., mucosal or systemic immunity). Uses of the compositions and methods provided by the present invention encompass human and non-human subjects and samples from those subjects, and also encompass research applications using these subjects. Compositions and methods of the present invention are also useful in studying and optimizing albumin variant, antigenic units, and other components and for screening for new components. Thus, it is not intended that the present invention be limited to any particular subject and/or application setting.

The present invention further provides kits comprising the vaccine compositions comprised herein. In some embodiments, the kit includes all of the components necessary, sufficient or useful for administering the vaccine. For example, in some embodiments, the kits comprise devices for administering the vaccine (e.g., needles or other injection devices), temperature control components (e.g., refrigeration or other cooling components), sanitation components (e.g., alcohol swabs for sanitizing the site of injection) and instructions for administering the vaccine.

Nucleic Acid Based Vaccines—Nucleic Acids Encoding a Protein Depicted in SEQ ID NO:1 or a Protein with an Amino Acid Identity of at Least 48% Identity with SEQ ID NO: 1

In particular embodiments the invention also relates to nucleic acid comprising a sequence which encodes a protein depicted in SEQ ID NO:1 or a nucleic acid sequence which encodes a protein with an amino acid identity of at least 48% identity with SEQ ID NO: 1. Nucleic acid according to the invention can take various forms (e.g. single-stranded, double-stranded, vectors etc.). Nucleic acids of the invention may be circular or branched, but will generally be linear. The nucleic acids used in the invention are preferably provided in purified or substantially purified form i.e. substantially free from other nucleic acids (e.g. free from naturally-occurring nucleic acids), particularly from other parasite or host cell nucleic acids, generally being at least about 50% pure (by weight), and usually at least about 90% pure.

Nucleic acids may be prepared in many ways e.g. by chemical synthesis (e.g. phosphoramidite synthesis of DNA) in whole or in part, by digesting longer nucleic acids using nucleases (e.g. restriction enzymes), by joining shorter nucleic acids or nucleotides (e.g. using ligases or polyrnerases), from genomic or cDNA libraries, etc.

The term “nucleic add” in general means a polymeric form of nucleotides of any length, which contain deoxyribonucleotides, ribonucleotides, and/or their analogs. It includes DNA, RNA, DNA/RNA hybrids. It also includes DNA or RNA analogs, such as those containing modified backbones (e.g. peptide nucleic adds (PNAs) or phosphorothioates) or modified bases. Thus, the nucleic acid of the disclosure includes mRNA, DNA, cDNA, recombinant nucleic acids, branched nucleic acids, plasmids, vectors, etc. Where the nucleic acid takes the form of RNA, it may or may not have a 5′ cap.

The nucleic acids of the invention comprise a sequence which encodes a protein depicted in SEQ ID NO:1 or a protein with an amino acid identity of at least 48% identity with SEQ ID NO: 1 (the latter serving as the antigen). Typically, the nucleic acids of the invention will be in recombinant form. For example, the nucleic acid may comprise one or more heterologous nucleic acid sequences (e.g. a sequence encoding another antigen and/or a control sequence such as a promoter or an internal ribosome entry site) in addition to the sequence encoding a protein depicted in SEQ ID NO:1 or a protein with an amino acid identity of at least 48% identity with SEQ ID NO: 1. The nucleic acid may be part of a vector i.e. part of a nucleic acid construct designed for transduction/transfection of one or more cell types. Vectors may be, for example, “expression vectors” which are designed for expression of a nucleotide sequence in a host cell, or “viral vectors” which are designed to result in the production of a recombinant virus or virus-like particle.

Alternatively, or in addition, the sequence or chemical structure of the nucleic acid maybe modified compared to a naturally-occurring sequence which encodes a protein depicted in SEQ ID NO:1 or a protein with an amino acid identity of at least 48% identity with SEQ ID NO: 1. The sequence of the nucleic acid molecule may be modified, e.g. to increase the efficacy of expression or replication of the nucleic acid, or to provide additional stability or resistance to degradation. For example, the sequence of the nucleic acid molecule may be codon optimized for expression in a desired host, such as a mammalian (e.g. human) cell. Such modification with respect to codon usage may increase translation efficacy and half-life of the nucleic acid. A poly A tail (e.g., of about 30 adenosine residues or more) may be attached to the 3′ end of the RNA to increase its half-life. The 5′ end of the RNA may be capped with a modified ribonucleotide with the structure m7G (5′) ppp (5′) N (cap 0 structure) or a derivative thereof, which can be incorporated during RNA synthesis or can be enzymatically engineered after RNA transcription (e.g., by using Vaccinia Virus Capping Enzyme (VCE) consisting of mRNA triphosphatase, guanylyl-transferase and guanine-7-methytransferase, which catalyzes the construction of N7-monomethylated cap 0 structures). Cap 0 structure plays an important role in maintaining the stability and translational efficacy of the RNA molecule. The 5′ cap of the RNA molecule may be further modified by a 2′-O-Methyltransferase which results in the generation of a cap 1 structure (m7Gppp [m2′-O] N), which may further increase translation efficacy.

The nucleic acids may comprise one or more nucleotide analogs or modified nucleotides. As used herein, “nucleotide analog” or “modified nucleotide” refers to a nucleotide that contains one or more chemical modifications (e.g., substitutions) in or on the nitrogenous base of the nucleoside (e.g., cytosine (C), thymine (T) or uracil (U)), adenine (A) or guanine (G)). A nucleotide analog can contain further chemical modifications in or on the sugar moiety of the nucleoside (e.g., ribose, deoxyribose, modified ribose, modified deoxyribose, six-membered sugar analog, or open-chain sugar analog), or the phosphate. The preparation of nucleotides and modified nucleotides and nucleosides are well-known in the art and many modified nucleosides and modified nucleotides are commercially available.

A composition as disclosed herein comprising a nucleic acid sequence which encodes a protein depicted in SEQ ID NO:1 or a protein with an amino acid identity of at least 48% identity with SEQ ID NO: 1 may be a nucleic acid-based vaccine. A further composition comprising a nucleic acid sequence which encodes one or more additional parasite antigens may also be provided as a nucleic acid-based vaccine.

The nucleic acid may, for example, be RNA (i. e. an RNA-based vaccine) or DNA (i. e. a DNA-based vaccine, such as a plasmid DNA vaccine), In certain embodiments, the nucleic acid-based vaccine is an RNA-based vaccine. In certain embodiments, the RNA-based vaccine comprises a self-replicating RNA molecule. The self-replicating RNA molecule may be an alphavirus-derived RNA replicon.

Self-replicating RNA molecules are well known in the art and can be produced by using replication elements derived from, e.g., alphaviruses, and substituting the structural viral proteins with a nucleotide sequence encoding a protein of interest. A self-replicating RNA molecule is typically a +-strand molecule which can be directly translated after delivery to a cell, and this translation provides an RNA-dependent RNA polymerase which then produces both antisense and sense transcripts from the delivered RNA. Thus, the delivered RNA leads to the production of multiple daughter RNAs. These daughter RNAs, as well as collinear subgenomic transcripts, may be translated themselves to provide in situ expression of an encoded antigen (i.e. a protein depicted in SEQ ID NO:1 or a protein with an amino acid identity of at least 48% identity with SEQ ID NO: 1), or may be transcribed to provide further transcripts with the same sense as the delivered RNA which are translated to provide in situ expression of the antigen. The overall result of this sequence of transcriptions is a huge amplification in the number of the introduced replicon RNAs and so the encoded antigen becomes a major polypeptide product of the cells.

The nucleic acid-based vaccine may comprise a viral or a non-viral delivery system. The delivery system (also referred to herein as a delivery vehicle) may have adjuvant effects which enhance the immunogenicity of the encoded antigen depicted in SEQ ID NO:1 or a protein with an amino acid identity of at least 48% identity with SEQ ID NO: 1. For example, the nucleic acid molecule may be encapsulated in liposomes, non-toxic biodegradable polymeric microparticles or viral replicon particles (VRPs) or complexed with particles of a cationic oil-in-water emulsion. In some embodiments, the nucleic acid-based vaccine comprises a cationic nano-emulsion (CNE) delivery system or a lipid nanoparticle (LNP) delivery system. Alternatively, the nucleic acid-based vaccine may comprise viral replicon particles. In other embodiments, the nucleic acid-based vaccine may comprise a naked nucleic acid, such as naked RNA (e.g. mRNA), but delivery via LNPs is preferred.

Antibodies Directed to SEQ ID NO: 1 and Proteins with an Amino Acid Identity of at Least 48% with SEQ ID NO: 1.

In another aspect, the invention relates to an antibody which specifically binds to a protein depicted in SEQ ID NO:1 or specifically bind to a protein with an amino acid identity of at least 48% identity with SEQ ID NO: 1.

An antibody that “specifically binds” to a protein depicted in SEQ ID NO:1 or a protein with an amino acid identity of at least 48% identity with SEQ ID NO: 1 is an antibody that binds this antigen with greater affinity and/or avidity than it binds to other parasite or non-parasite antigens. For example, the antibody which specifically binds to a protein depicted in SEQ ID NO:1 or a protein with an amino acid identity of at least 48% identity with SEQ ID NO: 1 (id est the antigen) may bind the parasite antigen with greater affinity and/or avidity than it binds to for example human serum albumin (HSA).

As used herein, the term “antibody” includes full-length or whole antibodies (i.e. antibodies in their substantially intact form), antibody fragments such as F(ab′)2, F(ab) and Fab′-SH fragments, Fv fragments (non-covalent heterodimers), single-chain antibodies such as single chain Fv molecules (scFv) or those derived from camelids and sharks (e.g. heavy chain antibodies), single-domain antibodies (dAbs), VHH's or nanobodies, diabodies, minibodies, oligobodies, etc. The term “antibody” does not imply any particular origin, and includes antibodies obtained through non-conventional processes, such as phage display. All of the antibodies will comprise the antigen binding site of a full-length or whole antibody and thus retain the ability of bind antigen. Thus, the term “antibody” includes antigen-binding fragments of full-length or whole antibodies. The antibody is ideally a monoclonal antibody, or, alternatively, may be polyclonal. The antibody may be chimeric, humanized, or fully human. In compositions of the invention, polyclonal antibody, comprising one or more antibodies which specifically bind to a protein depicted in SEQ ID NO:1 or a protein with an amino acid identity of at least 48% identity with SEQ ID NO: 1, may be used. In some preferred embodiments, the composition comprises a polyclonal antibody, for example serum anti-parasite protein depicted in SEQ ID NO:1 or a protein with an amino acid identity of at least 48% identity with SEQ ID NO: 1. The polyclonal antibody may comprise IgG (e.g. purified serum IgG). The antibody may comprise a neutralizing antibody (i. e. an antibody which neutralizes the biological effects of the parasite protein depicted in SEQ ID NO:1 or a protein with an amino acid identity of at least 48% identity with SEQ ID NO: 1 in the subject).

The antibody is preferably provided in purified or substantially purified form. Typically, the antibody will be present in a composition that is substantially free of other polypeptides e.g. where less than 90% (by weight), usually less than 60% and more usually less than 50%) of the composition is made up of other polypeptides.

The antibodies can be of any isotype (e.g. IgA, IgG, IgM i.e. an α, γ or μ heavy chain), but will generally be IgG, Within the IgG isotype, antibodies may be IgG1, IgG2, IgG3 or IgG4 subclass. The antibody may have a κ or a λ light chain.

In a particular embodiment the antibodies are immunoglobulin single variable domain antibodies such as VHH's or nanobodies.

In another particular embodiment the VHH sequences are depicted in SEQ ID NO: 30-37.

The sequences of SEQ ID NO: 30 to 37 are depicted below. The CDR1, CDR2 and CDR3 sequences are underlined (in the order CDR1, CDR2 and CDR3).

(Nb 27)
SEQ ID NO: 30
QVQLQESGGGLVQPGGSLRLSCTTSRSILSIYAMGWYRQAPGKQRELVA
VITSGGSTYYADSVKGRFTISRDNAKNTLYLQMNSLKPEDTALYYCARG
GYGTSPQPSWGQGTQVTVSS
CDR1 of Nb27: SEQ ID NO: 38:
RSILSIYA
CDR2 of Nb27: SEQ ID NO: 39:
ITSGGST
CDR3 of Nb27: SEQ ID NO: 40:
ARGGYGTSPQPS
(Nb 32)
SEQ ID NO: 31
QVQLQESGGGLVQPGGSLRLSCAASGFTFSAYAMSWVRQAPGKGLEWVS
TIDSGGGSTYYADSVKGRFTISRDNAKKTLYLHMNSLKPEDTAVYYCAK
RGYSDYARHVWGQGTQVTVSS
CDR1 of Nb32: SEQ ID NO: 41:
GFTFSAYA
CDR2 of Nb32: SEQ ID NO: 42:
IDSGGGST
CDR3 of Nb32: SEQ ID NO: 43:
AKRGYSDYARHV
(Nb34)
SEQ ID NO: 32
QVQLQESGGGLVQPGGSLRLSCAASGFTFSNSPMNWVRQAPGKGLEWVS
AINSGGGSTYYADSVKGRFTISRDNAKNTLYLQMNSLKPEDTAVYYCAK
GGRTYYSGSYYSWGMDYWGKGTQVTVSS
CDR1 of Nb34: SEQ ID NO: 44:
GFTFSNSP
CDR2 of Nb34: SEQ ID NO: 45:
INSGGGST
CDR3 of Nb34: SEQ ID NO: 46:
AKGGRTYYSGSYYSWGMDY
(Nb 36)
SEQ ID NO: 33
QVQLQESGGGLVQPGGSLRLSCAASGFAFSFYWMNWVRQAPGKGLEWVS
AINSGGGSTYYADSVKGRFTISRDNAKNTLYLQMNSLKPEDTAVYYCAK
GIVQRGEYYGMDYWGKGTQVTVSS
CDR1 of Nb36: SEQ ID NO: 47:
GFAFSFYW
CDR2 of Nb36: SEQ ID NO: 48:
INSGGGST
CDR3 of Nb36: SEQ ID NO: 49:
AKGIVQRGEYYGMDY
(Nb 39)
SEQ ID NO: 34
QVQLQESGGGLVQPGGSLRLSCTASGSIFSGYAMGWYRQAPGKQRELVA
GITSGGSTIYAGSVKGRFTISRDNAKNTVYLQMNSLKPEDTAVYYCKNG
TYYSARDYWGQGTQVTVSS
CDR1 of Nb39: SEQ ID NO: 50:
GSIFSGYA
CDR2 of Nb39: SEQ ID NO: 51:
ITSGGST
CDR3 of Nb39: SEQ ID NO: 52:
KNGTYYSARDY
(Nb 41)
SEQ ID NO: 35
QVQLQESGGGLVQAGGSLRLSCAASGRTFSSYAMGWFRQAPGKEREFIA
LISWIGGSTAYADSVKGRFTISRDNAKNTLYLQMNSLKPEDTAVYYCAT
DRIGRHRGPGTQVTVSS
CDR1 of Nb41: SEQ ID NO: 53:
GRTFSSYA
CDR2 of Nb41: SEQ ID NO: 54:
ISWIGGST
CDR3 of Nb41: SEQ ID NO: 55:
ATDRIGRH
(Nb 53)
SEQ ID NO: 36
QVQLQESGGGSVQAGGSLRLSCSASGYTYSSHCTAWFRQAPGKEREALA
AINSRSGTTYYADSVKGRFTISQDNTKNTAYLQMNSLKPEDTATYFCAA
LSFGCTGPLASLGQGTQVTVSS
CDR1 of Nb53: SEQ ID NO: 56:
GYTYSSHC
CDR2 of Nb53: SEQ ID NO: 57:
INSRSGTT
CDR3 of Nb53: SEQ ID NO: 58:
AALSFGCTGPLAS
(Nb 58)
SEQ ID NO: 37
QVQLQESGGGLVQPGGSLRLSCAASGRSISSIYAMGWYRQAPGKQRELV
ARLTSGGSSNYADSVKGRFTITRDNAQNTVYLQMNSLKPEDTAVYYCNA
EPSRWWLDDDYWGQGTQVTVSS
CDR1 of Nb58: SEQ ID NO: 59:
GRSISSIYA
CDR2 of Nb58: SEQ ID NO: 60:
LTSGGSS
CDR3 of Nb58: SEQ ID NO: 61:
NAEPSRWWLDDDY

In a specific embodiment the invention provides an immunoglobulin single variable domain (ISVD) antibody specifically binding to a protein having an amino acid identity of at least 48% with SEQ ID NO: 1 wherein said ISVD comprises 4 framework regions (FR) and 3 complementarity determining regions (CDR) according to the following formula (1): FR1-CDR1-FR2-CDR2-FR3-CDR3-FR4 (1);

and wherein CDR1 consists of a sequence depicted in SEQ ID NO: 38, 51, 44, 47, 50, 53, 56 or 59; CDR2 consists of a sequence depicted in SEQ ID NO: 39, 42, 45, 48, 51, 54, 57 or 60; and CDR3 consists of a sequence depicted in SEQ ID NO: 40, 43, 46, 49, 52, 55, 58 or 61.

In another specific embodiment the invention provides an immunoglobulin single variable domain (ISVD) antibody specifically binding to a protein having an amino acid identity of at least 48% with SEQ ID NO: 1 wherein said ISVD comprises 4 framework regions (FR) and 3 complementarity determining regions (CDR) according to the following formula (1): FR1-CDR1-FR2-CDR2-FR3-CDR3-FR4 (1);

and wherein CDR1 consists of a sequence depicted in SEQ ID NO: 38, 51, 44, 47, 50, 53, 56 or 59 or sequences with at least 95% identity with SEQ ID NO: 38, 51, 44, 47, 50, 53, 56 or 59; CDR2 consists of a sequence depicted in SEQ ID NO: 39, 42, 45, 48, 51, 54, 57 or 60 or sequences with at least 95% identity with SEQ ID NO: 39, 42, 45, 48, 51, 54, 57 or 60; and CDR3 consists of a sequence depicted in SEQ ID NO: 40, 43, 46, 49, 52, 55, 58 or 61 or sequences with at least 95% identity with SEQ ID NO: 40, 43, 46, 49, 52, 55, 58 or 61.

In yet another embodiment the invention provides the ISVD sequences as herein disclosed for use as a medicament.

In yet another embodiment the invention provides humanized sequences of SEQ ID NO: 30 to 37.

In yet another embodiment the invention provides the ISVD sequences as herein disclosed for use to treat or to prevent a mammal suffering from an infection from a species of the Trypanosomatidae family.

In another particular embodiment the ISVD sequences as disclosed herein are fused to a half-life extension domain and/or to an Fc fusion such as an IgA or IgG Fc fusion.

In yet another particular embodiment the invention provides the ISVD sequences as herein disclosed for in vitro use to detect (or diagnose) a trypanosomatid infection in a sample derived from a mammal.

### EXAMPLES

1. On Silico Analysis Identified Q586B2 as a Novel T. brucei Protein Exhibiting Structural Homology with the T. cruzi Protein Q4D6Q6

In an attempt to identify a MIF homologue within AT, the Leishmania MIF protein sequence (Q4Q413, SEQ ID NO: 2) was blasted against the available trypanosome genome (Trypanosoma brucei brucei TREU 927) within the Wellcome Trust Sanger Institute database (http://www.sanger.ac.uk/resources/databases). However, no bona fide MIF homologue was detected in trypanosomes. We then only used the first 25 aa of the Q4Q413 protein for blasting and focused on proteins with typical MIF-like characteristics, i.e. an amino-terminal proline residue immediately following the initial methionine residue and a maximum size of 115 aa. This approach identified Tb927.6.4140, a gene located on chromosome 6, coding for a hypothetical unknown protein (Q586B2, SEQ ID NO: 1) with an expected size of 13 kDa (348 bp, 115 aa) and a pl of 6.06, which overall shows little sequence similarity to MIF. In accordance, the Protein Homology/analogY Recognition Engine Version 2.0 (Phyre²) (http://www.sbg.bio.ic.ac.uk/phyre2/html/page.cgi?id=index), that also analyzes structural homology, yielded no homology between Q586B2 and MIF. However, Phyre² predicts a high homology to Q4D6Q6 (SEQ ID NO: 3), a conserved kinetoplastid-specific protein from Trypanosoma cruzi (55% sequence identity and 100% structural homology confidence), and a weak probability of homology (16% sequence identity and 51.5% structural homology confidence) with 4-OT of the Archaean species Archaeoglobus fulgidus. Of note, the recently obtained crystal structure of Q4D6Q6, for which the function remains enigmatic, demonstrated a propeller-like tetramer consisting of 4 monomers that adopt a 1313013013 topology. Using AlphaFold predictions (https://www.alphafold.ebi.ac.uk) we predicted the structure of Q586B2 as a monomer based on i) the Per-residue confidence (pLDDT), which is a per-residue measure of local confidence on a scale from 0-100. pLDDT depicting regions with low pLDDT that may be unstructured; either intrinsically disordered or structured only in the context of a larger complex, and ii) Predicted Aligned Error (PAE) whereby a consistently low PAE at (x, y) suggests AlphaFold is confident about the relative domain positions and high PAE at (x, y) suggests the relative positions of the domains should not be interpreted. It seems that Q586B2 has a high structural homology with 04D606. Subsequently, a comparison was made between the known structure of 04D606 forming a homo-tetramer and Q586B2, whereby it seems that Q586B2 can also form a homo-tetramer conformation. Collectively, these data demonstrated that trypanosomes do not harbor a bona fide MIF homologue, but uncovered Q586B2 as a protein with structural homology with 04D606, a T. cruzi protein for which the function remains enigmatic.

2. Q586B2 is an Evolutionary Conserved Protein within the Related Families Trypanosomatidae and Bodonidae.

To chart the taxonomic distribution of proteins homologous to Q586B2 and thereby elucidate the evolutionary origin of the underlying gene family, we screened a wide range of eukaryote genomes in the NCBI and TriTrypDB databases using tBLASTn searches for the presence of Tb927.6.4140 paralogous genes. These searches confirm the presence of three paralogous genes (Tb927.6.4140, Tb927.2.2770 and Tb10.26.0680) in T. brucei brucei as well as in the (sub)species T. brucei equiperdum, T. brucei gambiense, T. congolense, T. evansi and T. Vivax and all investigated trypanosomatid genera. A fourth paralogous gene was discovered only in four other Trypanosoma species (T. cruzi, T. rangeli, T. conorhini, T. theileri), as well as in all investigated species of the trypanosomatid genera Leishmania, Angomonas, Crithidia, Endotrypanum, Herpetomonas, Leptomonas, Lotmaria, Paratrypanosoma, Phytomonas and Strigomonas and in Bodo saltans, a free-living (non-parasitic) species of the closely related order Bodonida (Lukes et al. 2014) (see FIG. 1, sequences are depicted in the sequence listing: SEQ ID NO: 3-29).

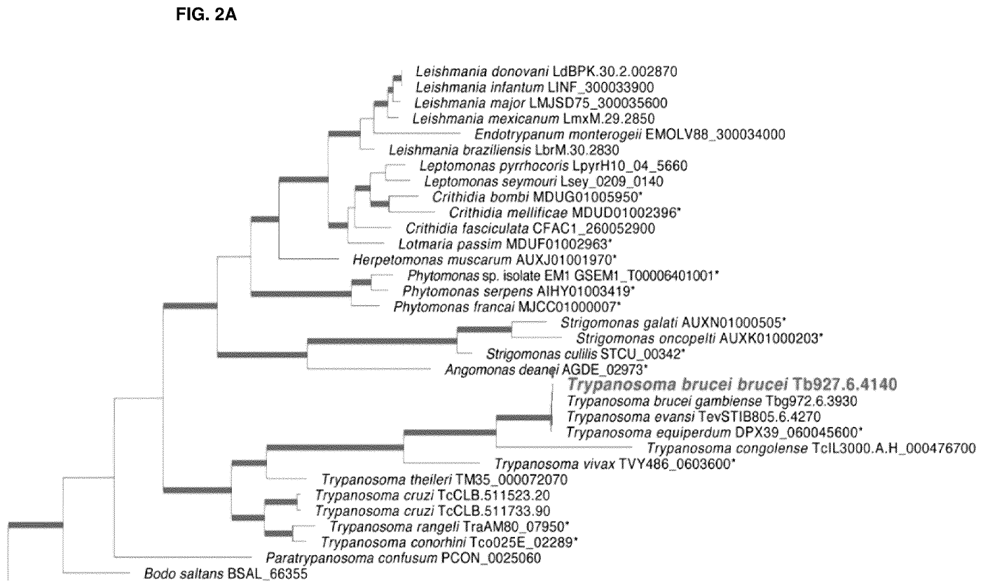

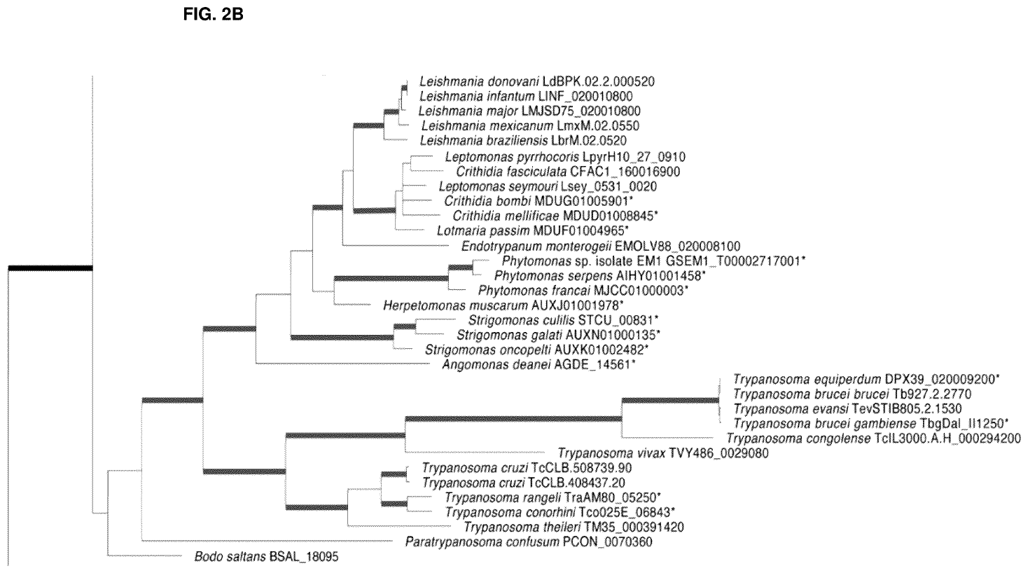

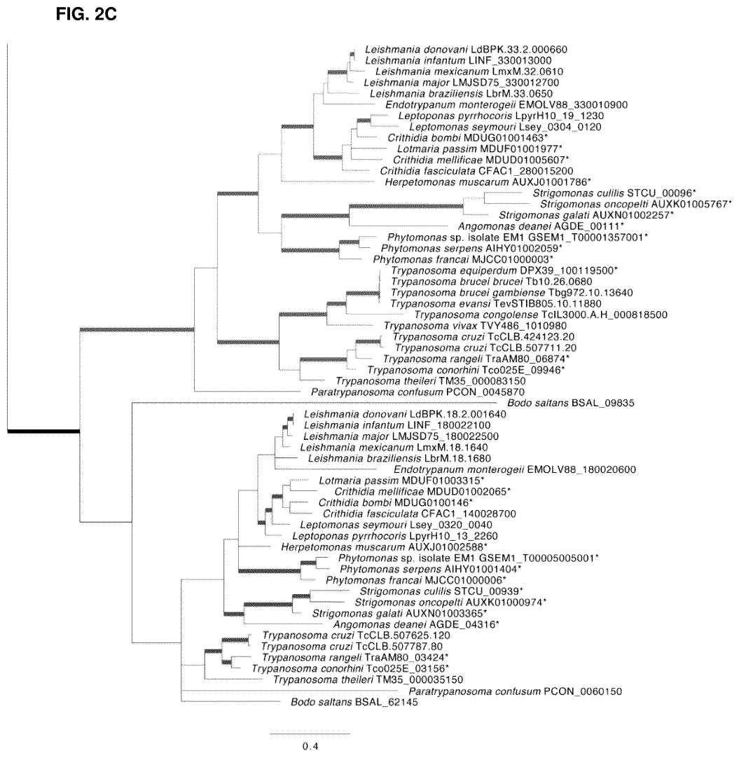

To investigate the evolutionary history of Q586B2 and its homologues, we conducted a Bayesian phylogenetic analysis based on an alignment of 126 gene sequences retrieved from the above mentioned taxa (FIGS. 2A-2C). In the absence of reliable outgroup sequences (homologous sequences for which a basal divergence with these 126 sequences is certain), the resulting consensus tree is a priori unrooted. However, a root position on any of the branches indicated with ‘R’ in FIGS. 2A-2C would implicate the most straightforward evolutionary scenario with a minimal number of gene duplication events and losses and is therefore the most likely under the maximum parsimony principle. The resulting tree is composed of four major gene clades, each including one of the four paralogues found in most species. This pattern indicates that a single ancestral gene underwent a series of duplication events (nodes indicated by ‘D’ in FIGS. 2A-2C) prior to the divergence of Bodo saltans and Trypanosomatidae, yielding a gene family of four paralogues that remained stable in most taxa. The presence of only three paralogues in T. brucei brucei and closely related (sub)species is explained by a single gene loss within Trypanosoma (marked by an ‘X’ in the purple gene Glade in FIG. 2C). The gene homologs encoding for Q586B2 (Tb927.6.4140, colored in red in FIG. 2A) are found deeply nested in one of these four gene clades, implying the presence of orthologous genes in all analysed trypanosomatid genera as well as in Bodo saltans. All genes encoding a protein with an amino-terminal proline are clustered in two of these four clades (red and blue in FIGS. 2A and 2B). Phyre² analysis of protein sequences of all four clades indicate with high confidence that they all share a tertiary structure similar to that of Q4D6Q6, implying the early rise and subsequent evolutionary conservation of this structure in this protein family.

Collectively, these data uncovered Q586B2 as an evolutionary conserved protein within trypanosomatids.

3. The Q586B2 Protein Exhibits Tautomerase Activity, is Present within Intracellular Vesicles and at Different Life Cycle Stages.

We next aimed to obtain more functional insights in the Q586B2 protein. Hence, we produced the protein recombinantly in E. coli WK6 cells by codon optimizing the Tb927.6.4140 gene and cloning it with an N-terminal His₆- and FLAG-tag, allowing removal of the tag after purification. Upon Q586B2 purification via IMAC and size exclusion chromatography it seems that the protein elutes as an oligomer with an estimated molecular weight of ^{˜}73 kDa, while a non-reducing SDS-PAGE revealed that the majority of the Q586B2 protein exists as a monomer but tends to form oligomers (dimers, trimers and tetramers). Since part of the Q586B2 protein showed resemblance to 4-OT, which belongs to the tautomerase superfamily, we first measured its tautomerase activity. Q586B2 indeed exhibits a moderate tautomerase activity, which is significantly lower than the prototypical tautomerase activity of MIF. Moreover, while MIF's tautomerase activity was inhibited by the small molecule ISO-1, this was not the case for Q586B2, suggesting structural differences in the active site.

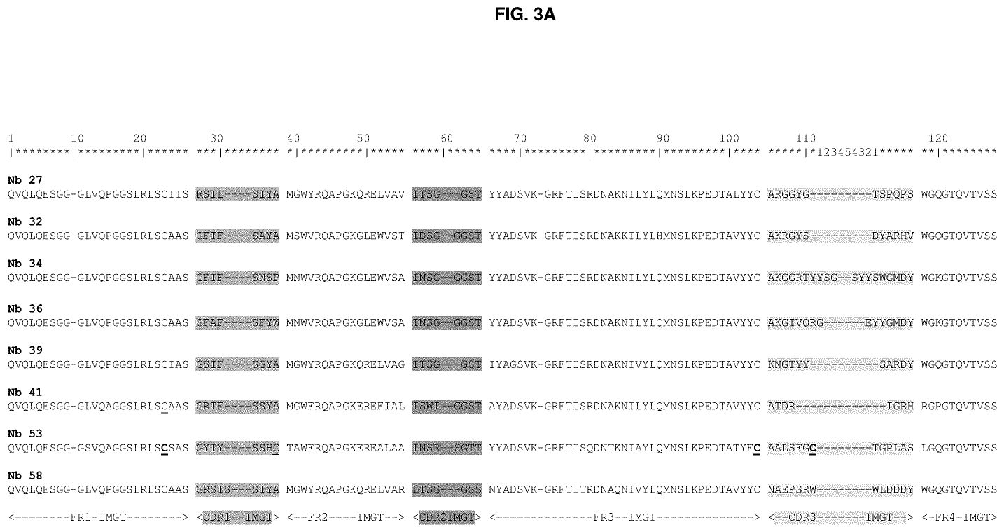

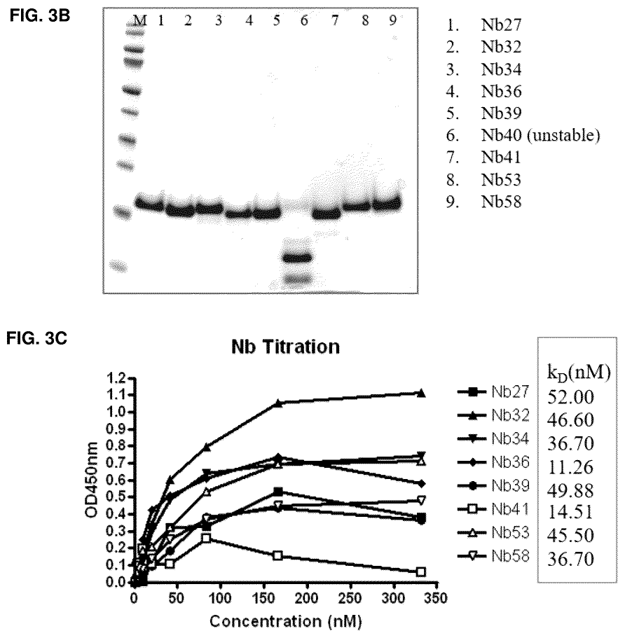

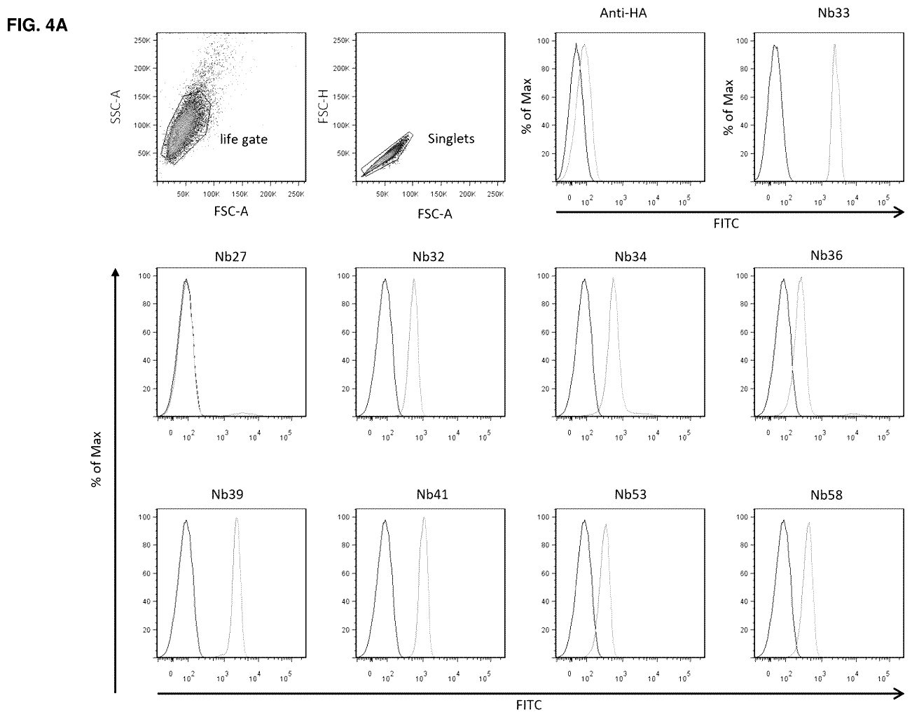

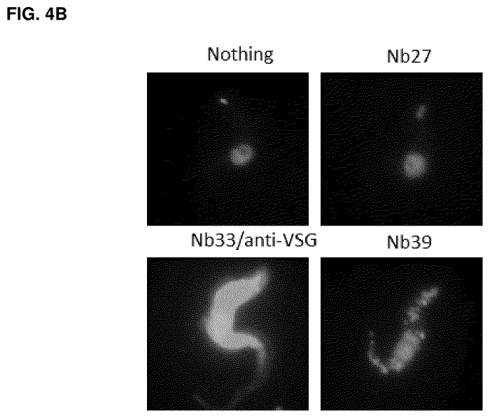

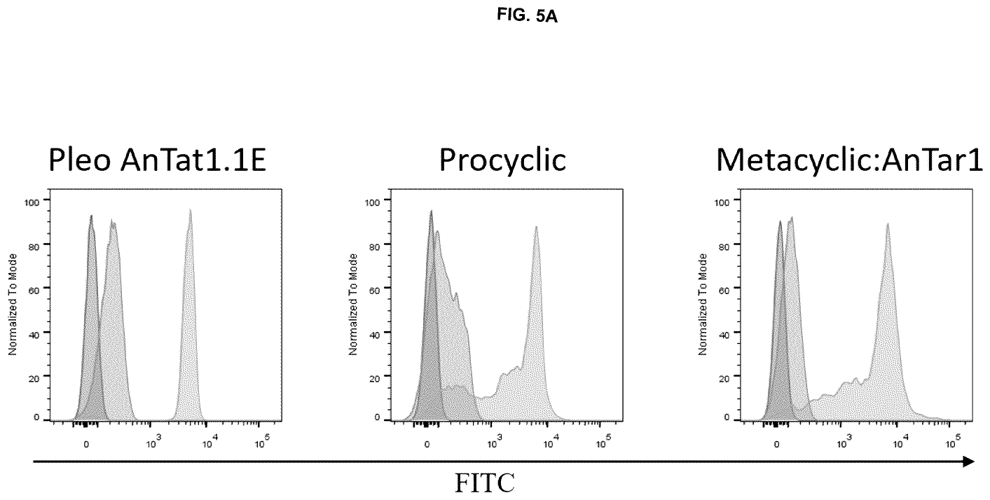

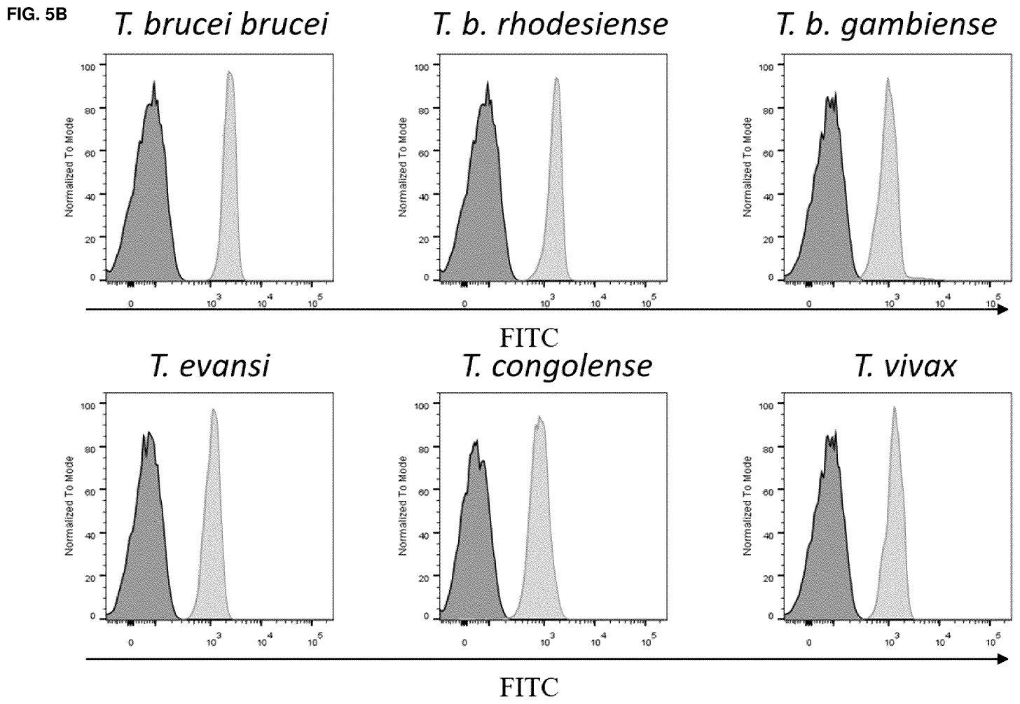

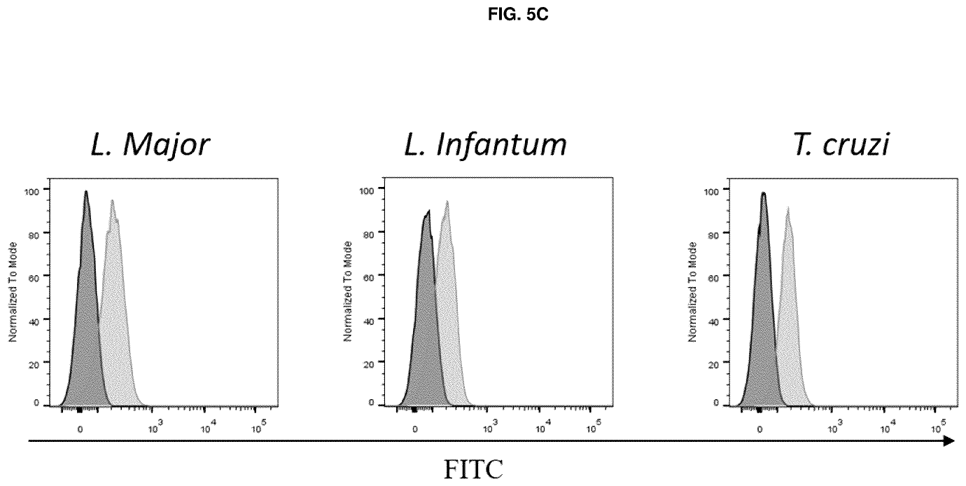

To generate novel research tools, we next used the recombinant Q586B2 protein for the generation of nanobodies (Nbs), which are the antigen-recognition domains of camelid heavy chain-only antibodies. Following immunization of a llama and screening of its Nb repertoire for specificity against the recombinant Q586B2 protein, 8 distinct Nbs could be retrieved exhibiting nM affinity for Q586B2 (FIG. 3). Using flow cytometry on purified T. brucei brucei parasites, these Nbs recognized their target only on fixed and permeabilized parasites, suggesting that Q586B2 is localized intracellularly (FIG. 4A). Nb39 consistently yielded the highest signal (MFI, median fluorescence intensity). Immunofluorescence microscopy with Nb39 confirmed that Q586B2 was localized in vesicles dispersed throughout the whole parasite (FIG. 4B). Interestingly, Q586B2 was expressed in the bloodstream stage as well as in the procyclic and metacyclic stages of T. brucei brucei, indicating that it is constitutively expressed throughout the entire life cycle of the parasite (FIG. 5A). Besides T. brucei brucei, also T. evansi, T. congolense, T. vivax and, importantly, the human pathogens T. b. gambiense and T. b. rhodesiense harbor this protein (FIG. 56), indicating that it is highly conserved among AT. These data also demonstrate that Nb39 recognizes a conserved region within the protein. Since other trypanosomatids belonging to the phylum Kinetoplastida were also suggested to harbor a Q586B2-like protein, Nb39 was tested on T. cruzi, L. major, and L. infantum, demonstrating the recognition of a protein in all three species, albeit at a lower level than in AT (FIG. 5C). Conversely, Q586B2 was not present in the parasitic protist P. falciparum belonging to the phylum Apicomplexa.

Collectively, Q586B2 was shown to harbor tautomerase activity, be localized in intracellular vesicles, expressed throughout the entire T. brucei brucei life cycle and to be present in most trypanosomatids belonging to the phylum Kinetoplastida.

4. Q586B2 is a Secreted Protein that is Able to Induce Early IL-10 Secretion by Myeloid Cells In Vivo, which Promotes T. brucei brucei Infection Onset.

The immunomodulatory effect of Q586B2 may be due to a direct effect on inflammatory cells, provided that this protein is secreted by the parasites. To test this possibility, we developed a Nb39-based sandwich ELISA that was able to detect recombinant Q586B2 in solution. Interestingly, Q586B2 was detected in the secretome of in vitro cultured WT parasites indicating that Q586B2 is released by WT parasites.

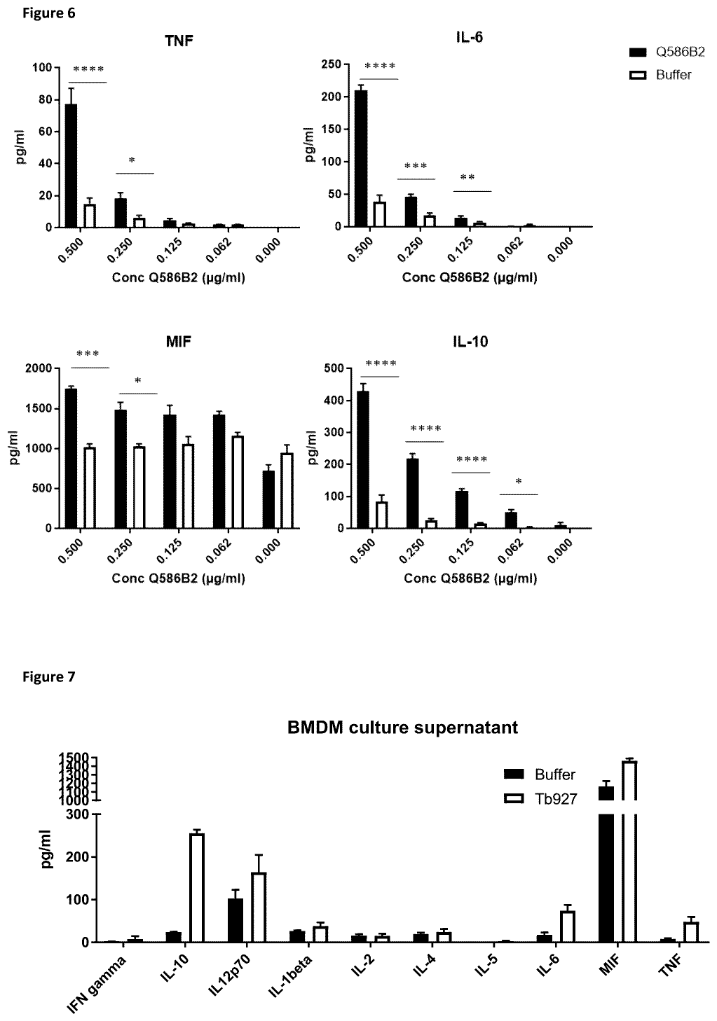

Macrophages are central regulators of inflammation, so we next assessed the direct effect of Q586B2 on these cells' ability to secrete pro- and anti-inflammatory cytokines (FIG. 6A). A 48 h culture of bone marrow-derived macrophages (BMDMs) with different concentrations of endotoxin-free Q586B2 protein (0.062; 0.125; 0.250; 0.500 μg/mL) resulted in a prominent induction of IL-10 secretion by even the lowest Q586B2 concentrations (8.4-fold induction at 0.250 μg/mL Q586B2) at which TNF, IL-6 and MIF are induced at much lower levels (2.9-fold induction of TNF, 2.6-fold induction of IL-6, 1.5-fold induction of MIF at 0.250 μg/mL Q586B2). Of note, at the highest concentration of Q586B2 (0.500 μg/mL) only TNF, IL-6, MIF and more strongly IL-10 were induced by BMDMs after 48 hours culturing (FIG. 7). These data suggest that Q586B2 may more potently induce an anti-inflammatory IL-10-driven response by macrophages.

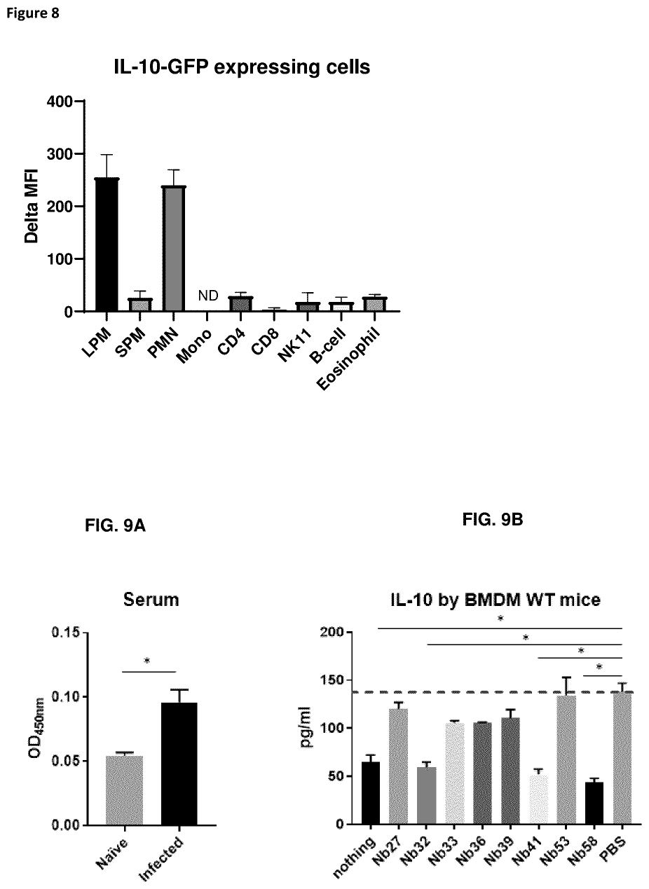

To assess whether Q586B2 is effectively able to trigger IL-10 production in vivo, we employed IL-10 reporter mice (VeRT-X mice). 18 hours after the intraperitoneal injection of 5 μg endotoxin-free Q586B2, IL-10 production was induced in resident large peritoneal macrophages (LPM) (DMFI as compared to PBS control=255.2), neutrophils (DMFI as compared to PBS control=240.3) and to a lesser extent CD4⁺ T cells (DMFI as compared to PBS control=29.17) (FIG. 8 for gating on LPM).

We conclude that T. brucei brucei parasites employ Q586B2 to trigger early IL-10 production, predominantly by myeloid cells at the site of infection, to increase the first peak of parasitemia.

5. Q586B2 as a Diagnostic and Therapeutic Target for Trypanosomatids

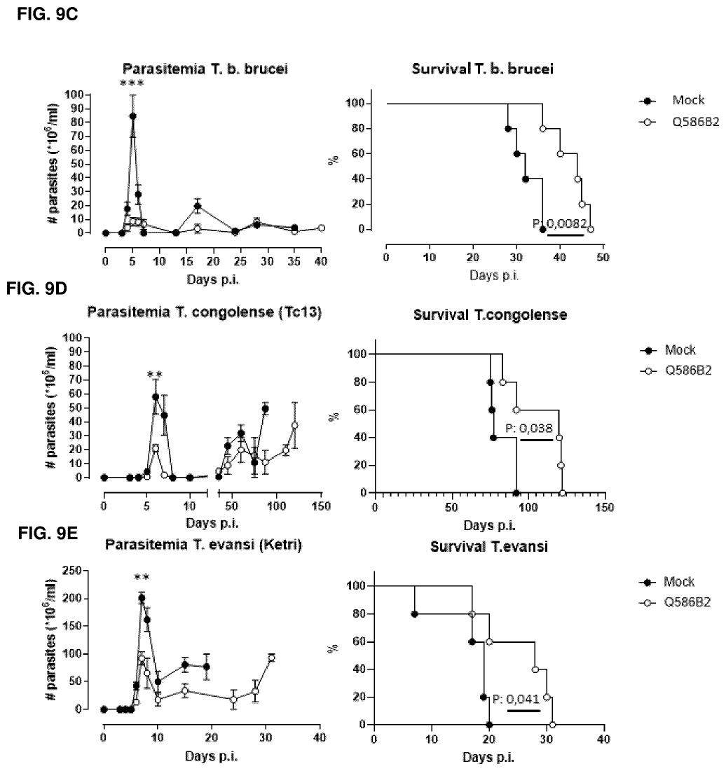

Since Q586B2 is produced early during the infection and promotes parasite establishment, this protein can be a diagnostic and therapeutic target. Employing the Nb39-based ELISA, Q586B2 could be detected in the serum of T. b. brucei infected mice as early as 6 days post infection (FIG. 9A). These data confirm the suitability of Q586B2 as a diagnostic biomarker and propose our in house-developed ELISA as a tool to detect it.

Next, the panel of anti-Q586B2 Nbs was tested for their ability to block the Q586B2-mediated IL-10 induction in macrophages. To this end, BMDMs were incubated with 1 μg/ml Q586B2 in the presence of 5 μg/ml anti-Q586B2 Nb. Several Nbs were found to significantly inhibit the IL-10-inducing potential of Q586B2, with Nb32, Nb41 and Nb58 being the most potent (FIG. 9B), thereby providing evidence for a therapeutic potential of these Nbs.

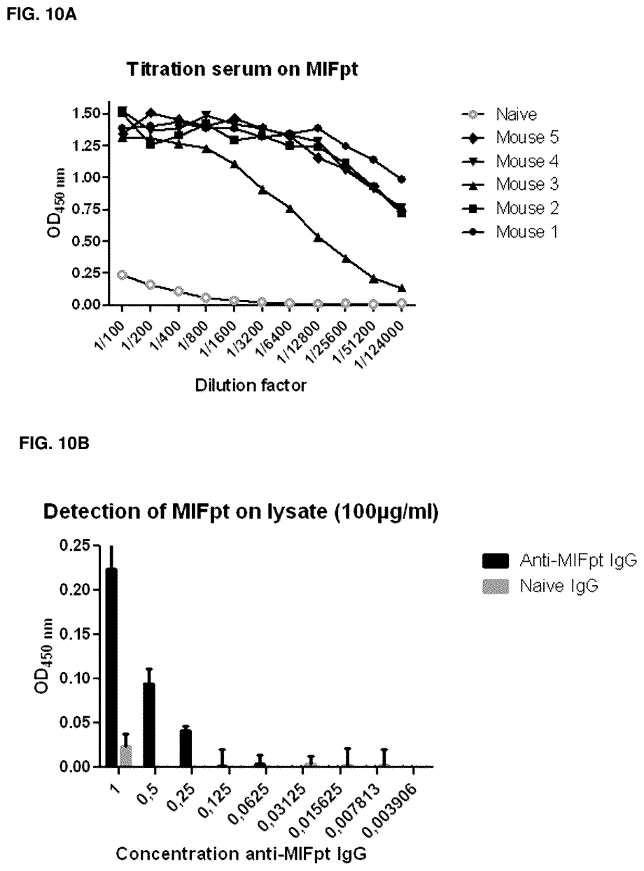

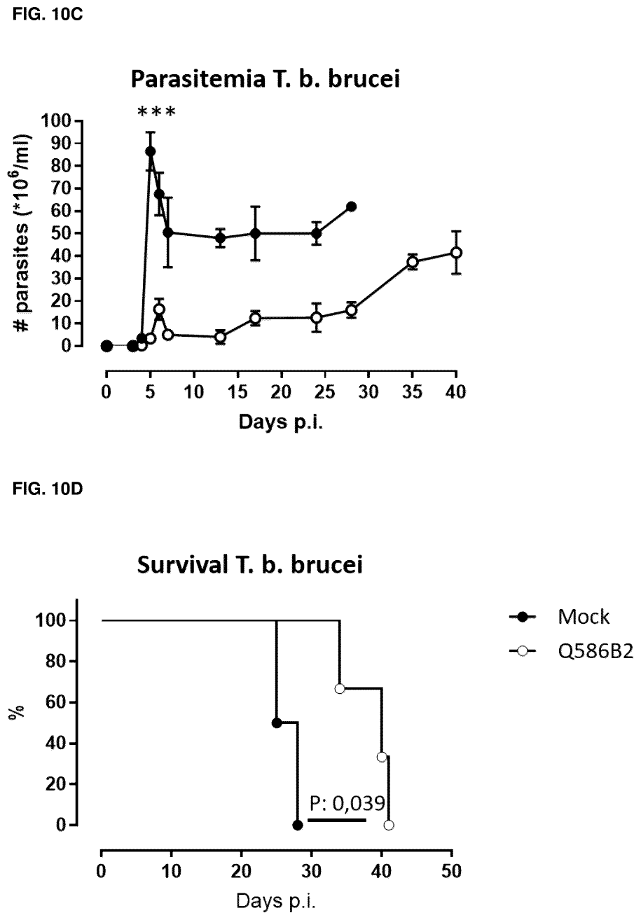

Given that Q586B2 is released into the circulation during infection, an alternative approach could be to prophylactically treat mice with Q586B2 aiming at immunomodulating the host response towards the parasite. Hence, we performed an intraperitoneal (i.p.) immunization schedule with Q586B2 in presence of adjuvant, repeated twice at 3 weeks interval, and this was referred to as Q586B2 pre-treatment. Control groups (mock-treated) included animals receiving only the adjuvant. The Q586B2 pre-treatment triggered a strong humoral immune response, whereby the purified anti-Q586B2 IgG were able to detect their native protein in parasite lysate in ELISA (FIGS. 10A and B, respectively). Subsequently, the Q586B2 pre-treated mice were challenged with wild type T. b. brucei parasites. Interestingly, Q586B2 pre-treated mice showed a significant reduction in first peak parasitemia, coinciding with a prolonged survival (mock-treated: 32.6+/−3.2 days versus Q586B2-treated: 42.4+/−4.4 days) (FIG. 9C). Surprisingly, this protective effect did not rely on the humoral immune response mounted, given that in B cell-deficient (μMT) mice this protective effect was maintained (FIG. 10C). Finally, since the protein was highly conserved within trypanosomatids, the Q586B2 pre-treatment strategy was evaluated for its cross-protective effect against other trypanosomatid species. Interestingly, Q586B2 pre-treated mice also showed a reduced first peak parasitemia upon challenge with T. congolense (Tc13) and T. evansi and coincided with a significant prolonged survival (Tc13: mock-treated: 82.4+/−8.0 days versus Q586B2-treated: 107.6+/−18.6 days and T. evansi: mock-treated: 16.4+/−5.3 days versus Q586B2-treated: 25.2+/−6.2 days) (FIGS. 9D and E, respectively).

6. Protection of Mammals Vaccinated with SEQ ID NO: 1 Against Leishmania major Infection

The immunogenic and prophylactic efficacy of recombinant Tb927.6.4140 protein (SEQ ID NO: 1) was evaluated in BALB/c mice challenged upon infection of Leishmania major. To this end, mice were immunized with recombinant Tb927.6.4140 and subsequently challenged with L. major (cutaneous leishmaniasis). L. major infections were initiated via transmission by infected Lutzomyia longipalpis sand flies. Twenty L. longipalpis sandflies infected with L. major (MHOM/SA/85/JISH118) promastigotes were placed in transmission cells which were attached on the right ears of sedated BALB/c mice for 20 minutes to allow natural vector-mediated parasite transmission.

For the vaccinated group: mice were injected intraperitoneally with 5 μg Tb927.6.4140 per mouse in complete Freud adjuvants (CFA). After three weeks, they were injected again with 5 μg Tb927.6.4140 per mouse in incomplete Freud adjuvants (IFA) and they received a third injection in IFA two weeks later. In addition, the effect of the vaccination vehicle alone was monitored as a separate group, thereto mice were injected in parallel with CFA or IFA respectively, without antigen, and subsequently infected. Infection was carried out 11 days after the third injection in IFA.

A control animal group not receiving any form of immunization, and subsequently infected, was included as well.

Each group contained 5 mice.

To evaluate vaccine efficacy, the L. major-infected mice were followed up with bioluminescent imaging (BLI) and the measured luminescence signals at the site of infection were compared between the groups. Despite the considerable variation in lesion onset between mice of the same group, most mice developed lesions by the end of the experiment (12 weeks (12 wpi)), except for the Tb927.6.4140-vaccinated group, where only 1 out of 4 mice developed a lesion. No statistical differences were noted in the BLI signals of the different groups throughout the entire experiment, but a statistical difference in average lesion size was noted at 12 wpi between the control and the Tb927.6.4140 group. To further evaluate this difference, RNA was extracted from the infected ears to compare the endpoint parasite burdens at the site of infection. When comparing the average Cq-values of the different treatment groups, however, no statistical difference could be demonstrated, indicating that even though no lesions had formed in the Tb927.6.4140-treated mice, comparable amounts of parasites were present at the infection site. The mouse that did develop a severe lesion in this group had the highest parasite burden.

Thus vaccination with Tb927.6.4140 did clearly show a statistically relevant inhibition in the development of lesions.

1. A vaccine composition, the composition comprising:
a protein having an amino acid identity of at least 48% over the full length of SEQ ID NO: 1, or a nucleic acid encoding the protein; and
an adjuvant.

2. The vaccine composition of claim 1, wherein the composition further comprises one or more proteins selected from SEQ ID NO: 3 to 29.

3. (canceled)

4. The vaccine composition of claim 1, wherein the nucleic acid is DNA.

5. The vaccine composition of claim 1, wherein the nucleic acid is RNA.

6. The vaccine composition of claim 1, further comprising a pharmaceutically acceptable carrier.

7. A method of treating a mammal for an infection of a pathogen species belonging to the family of Trypanosomatidae, the method comprising: administering to the mammal the vaccine composition of claim 1.

8. The method according to claim 7, wherein the pathogen species of the family of Trypanosomatidae is selected from the genera Trypanosoma or Leishmania.

9. The method according to claim wherein the Trypanosoma species is selected from the list consisting of Trypanosoma brucei gambiense, Trypanosoma brucei brucei, Trypanosoma brucei rhodesiense, Trypanosoma congolense, Trypanosoma vivax; Trypanosoma evansi, Trypanosoma equiperdum, and Trypanosoma cruzi.

10. The method according to claim 8, wherein the Leishmania species is Leishmania major or Leishmania infantum.
